(19) United States
(12) Patent Application Publication
(10) Pub. No.: US 2025/0092008 A1
(43) Pub. Date: Mar. 20, 2025

(54) PROCESS FOR MAKING AN EP4 ANTAGONIST

(71) Applicant: ONO PHARMACEUTICAL CO., LTD., Osaka-shi, Osaka (JP)

(72) Inventors: Ryoichi ARIHARA, Osaka (JP); Naoya SAGAWA, Osaka (JP); Michael Bryan HAY, Watchung, NJ (US); Sarah Evelyn STEINHARDT, Princeton, NJ (US); Steven R. WISNIEWSKI, Princeton, NJ (US); John Ryan COOMBS, Princeton, NJ (US); Gregory Louis BEUTNER, Princeton, NJ (US); Patricia Y. CHO, Gyeonggi-do (KR); Carlos A. GUERRERO, North Wales, PA (US); Miao YU, Princeton, NJ (US); Xuelei GUO, Princeton, NJ (US)

(73) Assignee: ONO PHARMACEUTICAL CO., LTD., Osaka-shi, Osaka (JP)

(21) Appl. No.: 18/294,296

(22) PCT Filed: Aug. 9, 2022
(86) PCT No.: PCT/JP2022/030354

§ 371 Date: Feb. 1, 2024

Related U.S. Application Data
(60) Provisional application No. 63/231,433, filed on Aug. 10, 2021.

(51) Int. Cl. C07D 311/96

(52) U.S. Cl. CPC C07D 311/96

(57) ABSTRACT

The present disclosure provides the methods for making 4-[4-cyano-2-({[(2′R,4S)-6-(isopropylcarbamoyl)-2,3-dihydrospiro[chromene-4,1′-cyclopropan]-2′-yl]carbonyl}amino)phenyl]butanoic acid. The present disclosure also generally relates to intermediates useful in the said methods.

### TECHNICAL FIELD

The present disclosure provides the methods for making 4-[4-cyano-2-({[(2′R,4S)-6-(isopropylcarbamoyl)-2,3-dihydrospiro[chromene-4,1′-cyclopropan]-2′-yl]carbonyl}amino)phenyl]butanoic acid. The present disclosure also generally relates to intermediates useful in the said methods.

### BACKGROUND ART

The EP₄ receptor is thought to be involved in inhibition of MCP-1 production from macrophages, inhibition of TNF-α, IL-2, and IFN-7 production from lymphocytes. This subtype is also believed to have involvement in anti-inflammation by enhanced IL-10 production, vasodilatation, angiogenesis, inhibition of elastic fiber formation, and regulation of MMP-9 expression. Other possible involvement of the EP₄ receptor includes immune control in cancer via myeloid derived suppressor cells, regulatory T cells, and natural killer cells. It is therefore thought that compounds that strongly bind to the EP₄ receptor and show antagonistic activity are useful for the prevention and/or treatment of diseases caused by EP₄ receptor activation, including, but not limited to, a cancer or an immune disease.

The compound 4-[4-cyano-2-({[(2′R,4S)-6-(isopropylcarbamoyl)-2,3-dihydrospiro[chromene-4,1′-cyclopropan]-2′-yl]carbonyl}amino)phenyl]butanoic acid (abbreviated as Compound (I) shown below) is an EP₄ antagonist and has demonstrated anti-cancer activity (see PTL 1).

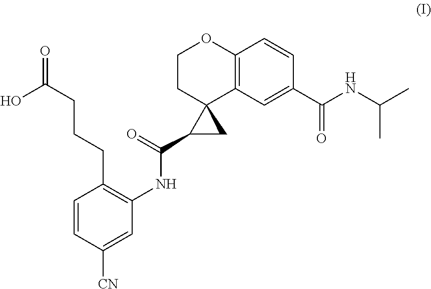 [SMILES: CC(C)NC(=O)c1ccc2c(c1)[C@]1(CCO2)C[C@H]1C(=O)Nc1cc(C#N)ccc1CCCC(=O)O]

Note that 4-[4-cyano-2-({[(2′R,4S)-6-(isopropylcarbamoyl)-2,3-dihydrospiro[chromene-4,1′-cyclopropan]-2′-yl]carbonyl}amino)phenyl]butanoic acid may also be named 4-[4-cyano-2-({(2′R,4S)-6-[(propan-2-yl)carbamoyl]-2,3-dihydrospiro[1-benzopyran-4,1′-cyclopropane]-2′-carbonyl}amino)phenyl]butanoic acid or 4-[4-cyano-2-({(1'S,2′R)-6-[(propan-2-yl)carbamoyl]-2,3-dihydrospiro[[1]benzopyran-4,1′-cyclopropane]-2′-carbonyl}amino)phenyl]butanoic acid.

Various production methods have been examined in providing the Compound (I) as a drug substance for a medicine. For example, a method for producing the Compound (I) in the Example 2-13 described in PTL 1 is known. However, the known method has been considered that the method is not suitable for large-scale production.

For purposes of large-scale production, there is a need for a high-yielding synthesis of the Compound (I) that is both efficient and cost-effective. As a result of intensive studies, the inventors have found a method for producing the Compound (I) that has both efficient and cost-effective.

### CITATION LIST

### Patent Literature

[PTL 1]
U.S. Pat. No. 10,077,247

### SUMMARY OF INVENTION

### Technical Problem

An object of the present disclosure is to provide a method for producing the Compound (I) that has both efficient and cost-effective.

### Solution to Problem

In one aspect, the present disclosure provides a process for making a compound of formula (2):

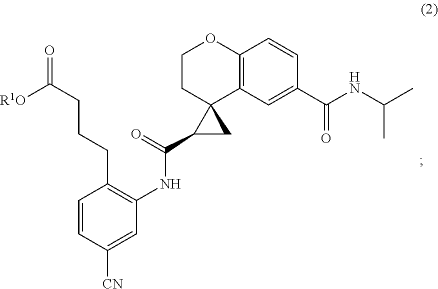

wherein R¹ is a C₁-C₆ alkyl group or a benzyl group; the process comprising: reacting a Compound (3):

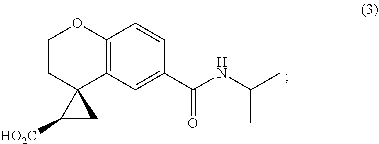 [SMILES: CC(C)NC(=O)c1ccc2c(c1)[C@]1(CCO2)C[C@H]1C(=O)O]

with a compound of formula (4a) or a salt thereof:

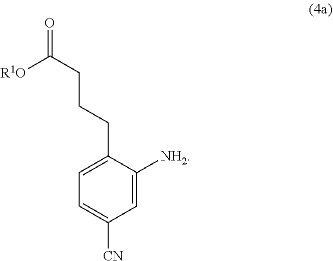

In another aspect, the reaction of the Compound (3) with the compound of formula (4a) or a salt thereof is conducted in the presence of a coupling agent. In another aspect, a compound of formula (4a) or a salt thereof is a compound of formula (4a). In another aspect, the coupling agent is selected from N,N,N′,N′-tetramethylchloroformamidinium hexafluorophosphate, 2,4,6-tripropyl-1,3,5,2,4,6-trioxatriphosphorinane-2,4,6-trioxide, and diphenyl phosphoryl chloride. In another aspect, the coupling agent is N,N,N′,N′-tetramethylchloroformamidinium hexafluorophosphate.

In another aspect, the process further comprises (i) reacting a compound of formula (13):

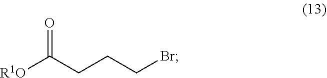

with bis(pinacolato)diboron in the presence of a first palladium catalyst and a first ligand to provide a compound of formula (14):

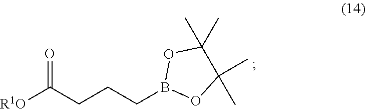

and (ii) treating the compound of formula (14) with a Compound (11):

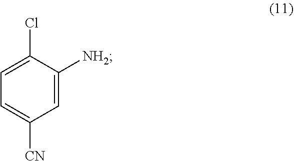

in the presence of a second palladium catalyst and a second ligand to provide the compound of formula (4a), wherein the second palladium catalyst and the second ligand may be formed complex,
wherein the compound of formula (4a) may be obtained a salt thereof.

In another aspect, the first palladium catalyst is selected from tris(dibenzylideneacetone)dipalladium(0), palladium acetate, and allyl palladium chloride dimer. In another aspect the first palladium catalyst is tris(dibenzylideneacetone)dipalladium(0).

In another aspect, the first ligand is selected from tricyclohexylphosphonium tetrafluoroborate, 2-dicyclohexylphophino-2′,6′-dimethoxybiphenyl (SPhos), triphenylphosphine, tri-ortho-tolylphospine, and butyldi-1-adamantylphosphine. In another aspect, the first ligand is tricyclohexylphosphonium tetrafluoroborate.

In another aspect, the second palladium catalyst is selected from Tetrakis(triphenylphosphine)palladium(0) (Pd(PPh₃)₄), palladium acetate, and allyl palladium chloride dimer. In another aspect, the second palladium catalyst is palladium acetate.

In another aspect, the second ligand is selected from di-tert-butylcyclohexylphosphine, 1,1-bis(dicyclohexylphosphino)ferrocene, 2-(dicyclohexylphosphino)3,6-dimethoxy-2′,4′,6′-triisopropyl-1,1′-biphenyl, di(1-adamantyl)-n-butylphosphine, and 1,2-ethanediylbis[dicyclohexyl]phosphine. In another aspect, the second ligand is 1,1-bis(dicyclohexylphosphino)ferrocene.

In another aspect, the second palladium catalyst and the second ligand are formed complexed. In another aspect, the complex is [1,1′-bis(dicyclohexylphosphino) ferrocene]dichloropalladium(II).

In another aspect, the present disclosure provides a process for making the Compound (I):

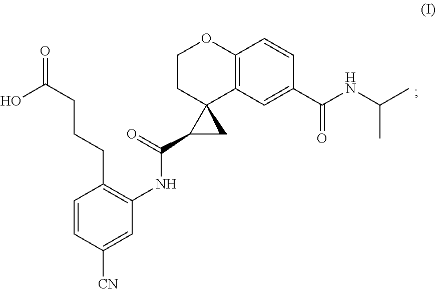 [SMILES: CC(C)NC(=O)c1ccc2c(c1)[C@]1(CCO2)C[C@H]1C(=O)Nc1cc(C#N)ccc1CCCC(=O)O]

comprising hydrolyzing or hydrocracking (hydrogenolysis) the compound of formula (2).

In another aspect, the present disclosure provides a process for making the Compound (I):

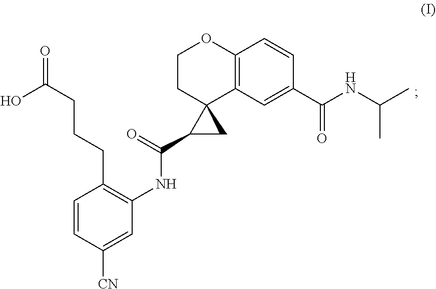 [SMILES: CC(C)NC(=O)c1ccc2c(c1)[C@]1(CCO2)C[C@H]1C(=O)Nc1cc(C#N)ccc1CCCC(=O)O]

comprising hydrolyzing the compound of formula (2).

In one aspect, the hydrolysis is conducted in the presence of a hydrolyzing agent selected from sodium hydroxide, lithium hydroxide, and potassium hydroxide. In another aspect, the hydrolyzing agent is sodium hydroxide.

In one aspect, the hydrogenolysis (hydrocracking) is conducted in the presence of a catalyst selected from palladium-carbon, palladium black, palladium hydroxide-carbon, platinum oxide and Raney nickel under hydrogen atmosphere at a normal pressure or under pressurization or in the presence of ammonium formate. In another aspect, the catalyst is palladium-carbon under hydrogen atmosphere at a normal pressure.

In another aspect, the present disclosure provides a process for making a Compound (3):

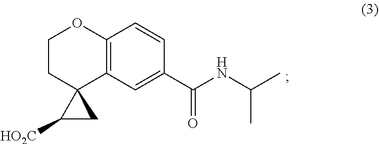 [SMILES: CC(C)NC(=O)c1ccc2c(c1)[C@]1(CCO2)C[C@H]1C(=O)O]

comprising:
(i) reacting a Compound (5):

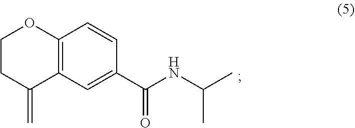 [SMILES: C=C1CCOc2ccc(C(=O)NC(C)C)cc21]

with a cyclopropanating agent in the presence of a catalyst and a chiral ligand to provide a compound of formula (5a):

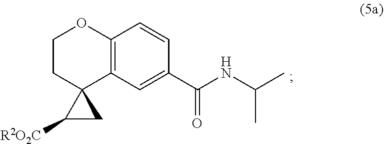

wherein R² is a C₁-C₆ alkyl group or a benzyl group;
and (ii) hydrolyzing or hydrocracking (hydrogenolysis) the compound of formula (5a).

In another aspect, the present disclosure provides a process for making a Compound (3):

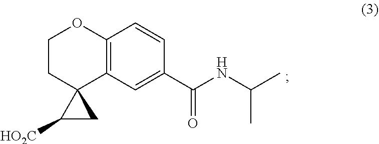 [SMILES: CC(C)NC(=O)c1ccc2c(c1)[C@]1(CCO2)C[C@H]1C(=O)O]

comprising:
(i) reacting a Compound (5):

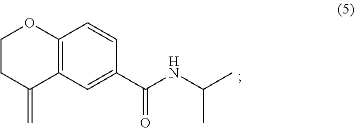 [SMILES: C=C1CCOc2ccc(C(=O)NC(C)C)cc21]

with a cyclopropanating agent in the presence of a catalyst and a chiral ligand to provide a compound of formula (5a):

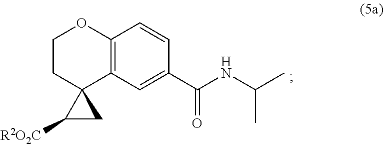

wherein R² is a C₁-C₆ alkyl group or a benzyl group;
and (ii) hydrolyzing the compound of formula (5a).

In another aspect, the cyclopropanating agent is selected from ethyl diazoacetate, methyl diazoacetate, n-butyl diazoacetate, benzyl diazoacetate, isopropyl diazoacetate, t-butyl diazoacetate. In another aspect, the cyclopropanating agent is ethyl diazoacetate. In another aspect, the catalyst is selected from dichloro(p-cymene)ruthenium(II) dimer, dibromo(p-cymene)ruthenium(II) dimer, and diiodo(p-cymene)ruthenium(II) dimer. In another aspect, the catalyst is dichloro(p-cymene)ruthenium(II) dimer. In another aspect, the chiral ligand is selected from (S,S)-2,2′-(2,6-pyridinediyl)bis(4-isopropyl-2-oxazoline), (−)-2,6-bis[(3aS,8aR)-3a,8a-dihydro-8H-indeno[1,2-d]oxazolin-2-yl]pyridine, and 2,6-bis[(4R,5R)-4-methyl-5-phenyl-2-oxazolinyl]pyridine. In another aspect, the chiral ligand is (S,S)-2,2′-(2,6-pyridinediyl)bis(4-isopropyl-2-oxazoline).

In another aspect, the hydrolysis in the preparation of compound (3) is conducted in the presence of a hydrolyzing agent selected from sodium hydroxide, lithium hydroxide, potassium hydroxide, tetraethylammonium hydroxide, potassium trimethylsilanolate, tetramethylammonium hydroxide, tetra-n-propylammonium hydroxide, tetra-n-butylammonium hydroxide and benzyltrimethylammonium hydroxide. In another aspect, the hydrolyzing agent is tetramethylammonium hydroxide. In another aspect, the hydrolyzing agent is sodium hydroxide.

In another aspect, the hydrogenolysis (hydrocracking) in the preparation of compound (3) is conducted in the presence of a catalyst selected from palladium-carbon, palladium black, palladium hydroxide-carbon, platinum oxide and Raney nickel under hydrogen atmosphere at a normal pressure or under pressurization or in the presence of ammonium formate. In another aspect, the catalyst is palladium-carbon under hydrogen atmosphere at a normal pressure.

In one aspect, the process further comprises (i) treating a Compound (6):

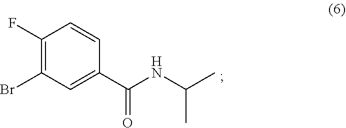

with 3-buten-1-ol to provide a Compound (7):

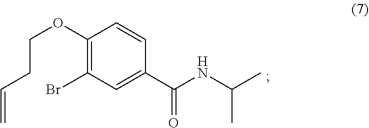 [SMILES: C=CCCOc1ccc(C(=O)NC(C)C)cc1Br]

and (ii) treating the obtained Compound (7) with a catalyst in the presence of a ligand to provide a Compound (5):

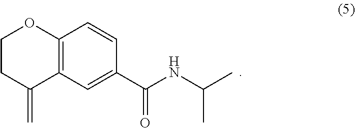 [SMILES: C=C1CCOc2ccc(C(=O)NC(C)C)cc21]

In another aspect, the compound (6) is treated with 3-buten-1-ol in the presence of a base. In another aspect, the base is selected from sodium tert-butoxide, potassium tert-butoxide, and lithium tert-butoxide. In another aspect, the base is potassium tert-butoxide.

In another aspect, the catalyst in the preparation of compound (5) is selected from palladium acetate and allyl palladium chloride dimer. In another aspect, the catalyst is palladium acetate. In another aspect, the ligand is selected from 1,2-bis(diphenylphosphino)benzene, 2-dicyclohexylphosphino-2′,6′-diisopropoxy-1,1′-biphenyl, (4-(N,N-dimethylamino)phenyl)di-tert-butyl phosphine, and butyl di-1-adamantylphosphine. In another aspect, the ligand is 1,2-bis(diphenylphosphino)benzene.

In another aspect, the present disclosure provides a process for making the Compound (I) comprising the below steps (i) to (ix);
(i) reacting a Compound (12):

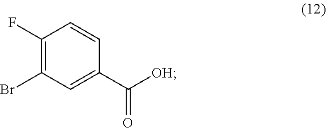 [SMILES: O=C(O)c1ccc(F)c(Br)c1]

with isopropylamine in the presence of a coupling agent to provide a Compound (6), (ii) treating a Compound (6) obtained in the step (i) with 3-buten-1-ol to provide a Compound (7),
(iii) treating a Compound (7) obtained in the step (ii) with a catalyst in the presence of a ligand to provide a Compound (5),
(iv) reacting a Compound (5) obtained in the step (iii) with a cyclopropanating agent in the presence of a catalyst and a chiral ligand to provide a compound of formula (5a) wherein R² is a C₁-C₆ alkyl group or a benzyl group,
(v) hydrolyzing the compound of formula (5a) obtained in the step (iv) to provide a Compound (3),
(vi) reacting a compound of formula (13) with bis(pinacolato)diboron in the presence of a first palladium catalyst and a first ligand to provide a compound of formula (14),
(vii) treating the compound of formula (14) obtained in the step (vi) with a Compound (11) in the presence of a second palladium catalyst and a second ligand to provide the compound of formula (4a), wherein the second palladium catalyst and the second ligand may be formed complex, wherein the compound of formula (4a) may be obtained a salt thereof,
(viii) reacting a Compound (3) obtained in the step (v) with a compound of formula (4a) or a salt thereof that is obtained in the step (vii) to provide a compound of formula (2), and (ix) hydrolyzing the compound of formula (2) obtained in the step (viii) to provide the Compound (I).

In another aspect, the present disclosure provides a process for making the Compound (I) comprising the below steps (ii) to (ix);
(ii) treating a Compound (6) with 3-buten-1-ol to provide a Compound (7),
(iii) treating a Compound (7) obtained in the step (ii) with a catalyst in the presence of a ligand to provide a Compound (5),
(iv) reacting a Compound (5) obtained in the step (iii) with a cyclopropanating agent in the presence of a catalyst and a chiral ligand to provide a compound of formula (5a) wherein R² is a C₁-C₆ alkyl group or a benzyl group,
(v) hydrolyzing the compound of formula (5a) obtained in the step (iv) to provide a Compound (3),
(vi) reacting a compound of formula (13) with bis(pinacolato)diboron in the presence of a first palladium catalyst and a first ligand to provide a compound of formula (14),
(vii) treating the compound of formula (14) obtained in the step (vi) with a Compound (11) in the presence of a second palladium catalyst and a second ligand to provide the compound of formula (4a), wherein the second palladium catalyst and the second ligand may be formed complex, wherein the compound of formula (4a) may be obtained a salt thereof,
(viii) reacting a Compound (3) obtained in the step (v) with a compound of formula (4a) or a salt thereof that is obtained in the step (vii) to provide a compound of formula (2), and (ix) hydrolyzing the compound of formula (2) obtained in the step (viii) to provide the Compound (I).

In another aspect, the present disclosure provides a process for making the Compound (I) comprising the below steps (iii) to (ix);
(iii) treating a Compound (7) with a catalyst in the presence of a ligand to provide a Compound (5),
(iv) reacting a Compound (5) obtained in the step (iii) with a cyclopropanating agent in the presence of a catalyst and a chiral ligand to provide a compound of formula (5a) wherein R² is a C₁-C₆ alkyl group or a benzyl group,
(v) hydrolyzing the compound of formula (5a) obtained in the step (iv) to provide a Compound (3),
(vi) reacting a compound of formula (13) with bis(pinacolato)diboron in the presence of a first palladium catalyst and a first ligand to provide a compound of formula (14),
(vii) treating the compound of formula (14) obtained in the step (vi) with a Compound (11) in the presence of a second palladium catalyst and a second ligand to provide the compound of formula (4a), wherein the second palladium catalyst and the second ligand may be formed complex, wherein the compound of formula (4a) may be obtained a salt thereof,
(viii) reacting a Compound (3) obtained in the step (v) with a compound of formula (4a) or a salt thereof that is obtained in the step (vii) to provide a compound of formula (2), and (ix) hydrolyzing the compound of formula (2) obtained in the step (viii) to provide the Compound (I).

In another aspect, the present disclosure provides a process for making the Compound (I) comprising the below steps (iv) to (ix);
(iv) reacting a Compound (5) with a cyclopropanating agent in the presence of a catalyst and a chiral ligand to provide a compound of formula (5a) wherein R² is a C₁-C₆ alkyl group or a benzyl group,
(v) hydrolyzing the compound of formula (5a) obtained in the step (iv) to provide a Compound (3),
(vi) reacting a compound of formula (13) with bis(pinacolato)diboron in the presence of a first palladium catalyst and a first ligand to provide a compound of formula (14),
(vii) treating the compound of formula (14) obtained in the step (vi) with a Compound (11) in the presence of a second palladium catalyst and a second ligand to provide the compound of formula (4a), wherein the second palladium catalyst and the second ligand may be formed complex, wherein the compound of formula (4a) may be obtained a salt thereof,
(viii) reacting a Compound (3) obtained in the step (v) with a compound of formula (4a) or a salt thereof that is obtained in the step (vii) to provide a compound of formula (2), and (ix) hydrolyzing the compound of formula (2) obtained in the step (viii) to provide the Compound (I).

In another aspect, the present disclosure provides a process for making the Compound (I) comprising the below steps (v) to (ix);
(v) hydrolyzing the compound of formula (5a) to provide a Compound (3),
(vi) reacting a compound of formula (13) with bis(pinacolato)diboron in the presence of a first palladium catalyst and a first ligand to provide a compound of formula (14),
(vii) treating the compound of formula (14) obtained in the step (vi) with a Compound (11) in the presence of a second palladium catalyst and a second ligand to provide the compound of formula (4a), wherein the second palladium catalyst and the second ligand may be formed complex, wherein the compound of formula (4a) may be obtained a salt thereof,
(viii) reacting a Compound (3) obtained in the step (v) with a compound of formula (4a) or a salt thereof that is obtained in the step (vii) to provide a compound of formula (2), and (ix) hydrolyzing the compound of formula (2) obtained in the step (viii) to provide the Compound (I).

In another aspect, the present disclosure provides a process for making the Compound (I) comprising the below steps (vi) to (ix);
(vi) reacting a compound of formula (13) with bis(pinacolato)diboron in the presence of a first palladium catalyst and a first ligand to provide a compound of formula (14),
(vii) treating the compound of formula (14) obtained in the step (vi) with a Compound (11) in the presence of a second palladium catalyst and a second ligand to provide the compound of formula (4a), wherein the second palladium catalyst and the second ligand may be formed complex, wherein the compound of formula (4a) may be obtained a salt thereof,
(viii) reacting a Compound (3) with a compound of formula (4a) or a salt thereof that is obtained in the step (vii) to provide a compound of formula (2), and (ix) hydrolyzing the compound of formula (2) obtained in the step (viii) to provide the Compound (I).

In another aspect, the present disclosure provides a process for making the Compound (I) comprising the below steps (vii) to (ix);
(vii) treating the compound of formula (14) with a Compound (11) in the presence of a second palladium catalyst and a second ligand to provide the compound of formula (4a), wherein the second palladium catalyst and the second ligand may be formed complex, wherein the compound of formula (4a) may be obtained a salt thereof,
(viii) reacting a Compound (3) with a compound of formula (4a) or a salt thereof that is obtained in the step (vii) to provide a compound of formula (2), and (ix) hydrolyzing the compound of formula (2) obtained in the step (viii) to provide the Compound (I).

In another aspect, the present disclosure provides a process for making a Compound (I) comprising the below steps;
reacting a Compound (3) with a compound of formula (4a) or a salt thereof to provide a compound of formula (2),
and hydrolyzing the compound of formula (2).

In another aspect, the present disclosure provides a process for making a Compound (I) comprising the below steps;
hydrolyzing the compound of formula (5a) to provide a Compound (3), reacting a Compound (3) with a compound of formula (4a) or a salt thereof to provide a compound of formula (2)
and hydrolyzing the compound of formula (2).

In another aspect, the present disclosure provides a process for making a Compound (I) comprising the below steps;
reacting a Compound (5) with a cyclopropanating agent in the presence of a catalyst and a chiral ligand to provide a compound of formula (5a),
hydrolyzing the compound of formula (5a) to provide a Compound (3),
reacting a Compound (3) with a compound of formula (4a) or a salt thereof to provide a compound of formula (2)
and hydrolyzing the compound of formula (2).

In another aspect, the present disclosure provides a process for making a Compound (I) comprising the below steps;
treating a Compound (7) with a catalyst in the presence of a ligand to provide a Compound (5)
reacting a Compound (5) with a cyclopropanating agent in the presence of a catalyst and a chiral ligand to provide a compound of formula (5a),
hydrolyzing the compound of formula (5a) to provide a Compound (3),
reacting a Compound (3) with a compound of formula (4a) or a salt thereof to provide a compound of formula (2)
and hydrolyzing the compound of formula (2).

In another aspect, R¹ is preferably methyl or ethyl. In another aspect, R¹ is more preferably ethyl.

In another aspect, R² is preferably methyl or ethyl. In another aspect, R² is more preferably ethyl.

Other aspects of the present disclosure may comprise suitable combinations of two or more of the aspects disclosed herein.

In another aspect, the present disclosure provides a compound of (1'S,2′R)-6-[(propan-2-yl)carbamoyl]-2,3-dihydrospiro[[1]benzopyran-4,1′-cyclopropane]-2′-carboxylic acid.

In another aspect, the present disclosure provides a compound of ethyl (1'S,2′R)-6-[(propan-2-yl)carbamoyl]-2,3-dihydrospiro[[1]benzopyran-4,1′-cyclopropane]-2′-carboxylate.

In another aspect, the present disclosure provides a compound of 4-methylidene-N-(propan-2-yl)-3,4-dihydro-2H-1-benzopyran-6-carboxamide.

In another aspect, the present disclosure provides a compound of 3-bromo-4-[(but-3-en-1-yl)oxy]-N-(propan-2-yl)benzamide.

### Advantageous Effects of Invention

The present disclosure provide a method for producing the Compound (I) that has both efficient and cost-effective.

### DESCRIPTION OF EMBODIMENTS

Unless otherwise indicated, any atom with unsatisfied valences is assumed to have hydrogen atoms sufficient to satisfy the valences.

As used in the present specification, the following terms have the meanings indicated:

The singular forms “a,” “an,” and “the” include plural referents unless the context dictates otherwise.

As used herein, the term “or” is a logical disjunction (i.e., and/or) and does not indicate an exclusive disjunction unless expressly indicated such as with the terms “either,” “unless,” “alternatively,” and words of similar effect.

The term “C₁-C₆ alkyl,” as used herein, refers to a group derived from a straight or branched chain saturated hydrocarbon containing from one to six carbon atoms. Examples of C₁-C₆ alkyl include, but are not limited to, methyl, ethyl, n-propyl, isopropyl, n-butyl, sec-butyl, tert-butyl, isobutyl, pentyl, 1-methyl butyl, 2-methyl butyl, 3-methyl butyl, 1,1-dimethyl propyl, 1,2-dimethyl propyl, 2,2-dimethyl propyl, hexyl, 1-methyl pentyl, 2-methyl pentyl, 3-methyl pentyl, 4-methyl pentyl, 1,1-dimethyl butyl, 1,2-dimethyl butyl, 1,3-dimethyl butyl, 2,2-dimethyl butyl, 2,3-dimethyl butyl, 1-methyl-1-ethyl propyl, 2-methyl-2-ethyl propyl, 1-ethyl butyl, and 2-ethyl butyl.

The term “coupling agent,” as used herein, refers to a reagent that facilitates the reaction of an amine and a carboxylic acid to form an amide bond. Examples of coupling agents include, but are not limited to, N,N,N,N′-tetramethylchloroformamidinium hexafluorophosphate, 2,4,6-tripropyl-1,3,5,2,4,6-trioxatriphosphorinane-2,4,6-trioxide, diphenylphosphinic chloride, diphenyl phosphoryl chloride, N,N,N,N′-tetramethylfluoroformamidinium hexafluorophosphate, N,N,N′,N′-tetramethyl-O-(1H-benzotriazol-1-yl)uronium hexafluorophosphate, benzotriazol-1-yl-oxytripyrrolidinophosphonium hexafluorophosphate, bis(2-oxo-3-oxazolidinyl)phosphinic chloride, 4-(4,6-dimethoxy-1,3,5-triazin-2-yl)-4-methylmorpholinium chloride, O-[(ethoxycarbonyl)cyanomethylenamino]-N,N,N′,N′-tetramethyluronium tetrafluoroborate, 1-ethyl-3-(3-dimethylaminopropyl)carbodiimide, and O—(N-succinimidyl)-N,N,N′,N′-tetramethyluronium hexafluorophosphate.

The term “cyclopropanating agent,” as used herein, refers to a reagent that is used to convert an alkene to a cyclopropyl ring. Examples of cyclopropanating agents include, but are not limited to, C₁-C₆ alkyl diazoacetate and benzyl diazoacetate. Specific examples thereof include ethyl diazoacetate, methyl diazoacetate, n-butyl diazoacetate, benzyl diazoacetate, isopropyl diazoacetate, t-butyl diazoacetate, n-propyl diazoacetate, sec-butyl diazoacetate, isobutyl diazoacetate, n-pentyl diazoacetate, n-hexyl diazoacetate, 1-methyl butyl diazoacetate, 2-methyl butyl diazoacetate, 3-methyl butyl diazoacetate, 1,1-dimethyl propyl diazoacetate, 1,2-dimethyl propyl diazoacetate, 2,2-dimethyl propyl diazoacetate, 1-methyl pentyl diazoacetate, 2-methyl pentyl diazoacetate, 3-methyl pentyl diazoacetate, 4-methyl pentyl diazoacetate, 1,1-dimethyl butyl diazoacetate, 1,2-dimethyl butyl diazoacetate, 1,3-dimethyl butyl diazoacetate, 2,2-dimethyl butyl diazoacetate, 2,3-dimethyl butyl diazoacetate, 1-methyl-1-ethyl propyl diazoacetate, 2-methyl-2-ethyl propyl diazoacetate, 1-ethyl butyl diazoacetate, and 2-ethyl butyl diazoacetate. Preferred examples include ethyl diazoacetate, methyl diazoacetate, n-butyl diazoacetate, benzyl diazoacetate, isopropyl diazoacetate, t-butyl diazoacetate, n-propyl diazoacetate, sec-butyl diazoacetate, isobutyl diazoacetate, n-pentyl diazoacetate, and n-hexyl diazoacetate. More preferred examples include ethyl diazoacetate, methyl diazoacetate, n-butyl diazoacetate, benzyl diazoacetate, isopropyl diazoacetate, and t-butyl diazoacetate. Most preferred example is ethyl diazoacetate.

The term “hydrolyzing agent,” as used herein, refers to a reagent that facilitates the conversion of an ester to a carboxylic acid. Examples of hydrolyzing agents include, but are not limited to, sodium hydroxide, lithium hydroxide, potassium hydroxide, tetramethylammonium hydroxide, cesium hydroxide, tetra-n-butylammonium hydroxide, tetra-n-propylammonium hydroxide, tetraethylammonium hydroxide, benzyltrimethylammonium hydroxide, sodium trimethylsilanolate, potassium trimethylsilanolate, sodium carbonate, sodium bicarbonate, and postassium phosphate.

All of the processes in the present disclosure can be conducted as continuous processes. The term “continuous process,” as used herein, represents steps conducted without isolation of the intermediate.

The following synthetic schemes illustrate methods by which the compounds of the present disclosure can be prepared. Starting materials can be obtained from commercial sources or prepared by well-established literature methods known to those of ordinary skill in the art.

Abbreviations used in the schemes and in the examples which follow are well-known to those skilled the art. Some of the abbreviations used are as follows: MTBE for methyl tert-butyl ether; MSA for methanesulfonic acid; NMT for not more than; NLT for not less than; h for hours; min for minutes; THF for tetrahydrofuran; MeTHF or 2-MeTHF for 2-methyltetrahydrofuran, DMSO for dimethylsulfoxide; aq for aqueous; equiv for equivalents; LR for limiting reagent; iPr-Pybox for 2,6-bis(4-isopropyl-2-oxazolin-2-yl)pyridine and RBF for round bottom flask.

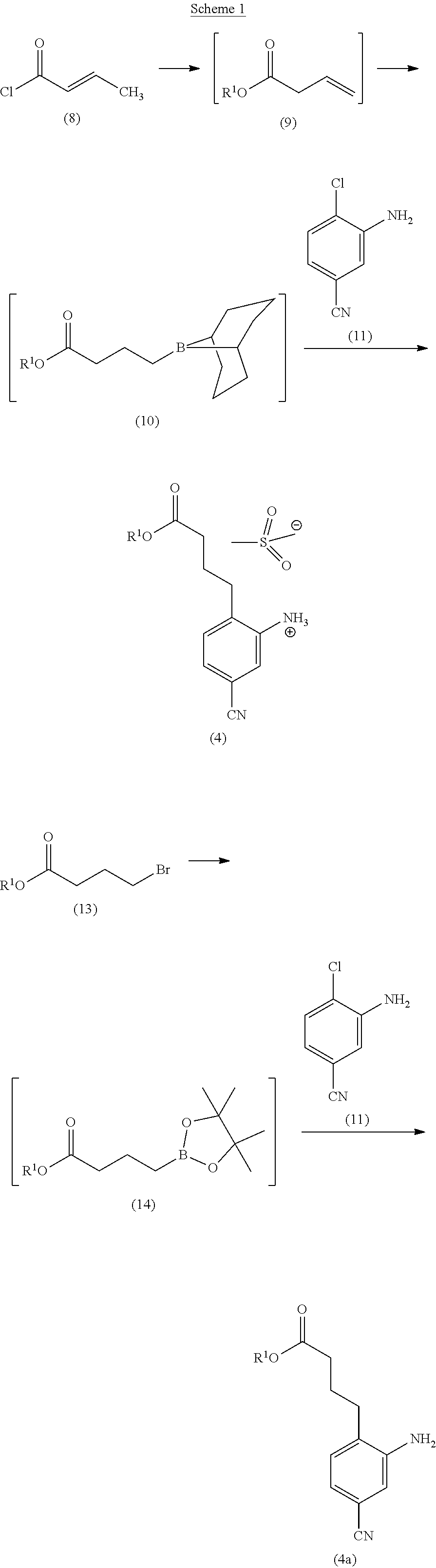

Scheme 1 illustrates the synthesis of compounds of formula (4) and (4a). Treatment of crotonyl chloride (Compound (8)) with a C₁-C₆ alcohol in the presence of a base results in the formation of ester (the compound of formula (9)) (See Bull. Chem. Soc. Japan 1967, 40, 1132-1239). Examples of bases that can be used in this reaction include, but are not limited to, triethylamine, tributylamine, N-ethylpiperidine, and dimethylcyclohexylamine. In one aspect the base is triethylamine. The resulting ester can be treated with 9-borabicyclo[3.3.1]nonane (9-BBN) and coupled with aryl halide (Compound (11)) in the presence of a palladium catalytic system to provide the Compound of formula (4a) which is then treated with MSA to provide the Compound of formula (4). Representative catalytic systems include, but are not limited to Tetrakis(triphenylphosphine)palladium(0) (Pd(PPh₃)₄), or palladium acetate or allyl palladium chloride dimer with a ligand such as 2-dicyclohexylphophino-2′,6′-dimethoxybiphenyl (SPhos), tricyclohexylphosphine, phenyldicyclohexylphosphine 1,1-bis(dicyclohexylphosphino)ferrocene, 2-dicyclohexylphosphino-2′,4′,6′-triisopropylbiphenyl. In one aspect, the catalytic system is palladium acetate and 2-dicyclohexylphosphino-2′,6′-dimethoxybiphenyl (SPhos). Treatment with methanesulfonic acid forms methanesulfonic acid salt (Compound of formula (4)). Alternative salts can be prepared in a similar manner, by reacting the free base of the Compound of formula (4) with an alternative acid.

Alternatively, a C₁-C₆ alkyl ester of 4-bromobutanoic acid (Compound of formula (13)) can be treated with bis(pinacolato)diboron in the presence of a palladium catalytic system to provide boronate (Compound of formula (14)) (See, J. Org. Chem, 2012, 77, 6629-6633). Examples of catalytic systems include tris(dibenzylideneacetone)dipalladium(0), palladium acetate, or allyl palladium chloride dimer with a ligand such as tricyclohexylphosphonium tetrafluoroborate, 2-dicyclohexylphophino-2′,6′-dimethoxybiphenyl (SPhos), triphenylphosphine, tri-ortho-tolylphospine, and butyldi-1-adamantylphosphine. In one aspect, the catalytic system is tris(dibenzylideneacetone)dipalladium(0) and tricyclohexylphosphonium tetrafluoroborate.

Boronate (Compound of formula (14)) can be coupled with aryl halide (Compound (11)) in the presence of a palladium catalytic system to provide the Compound of formula (4a). Representative catalytic systems include, but are not limited to Tetrakis(triphenylphosphine)palladium(0) (Pd(PPh₃)₄), or palladium acetate or allyl palladium chloride dimer with a ligand such as 1,1-bis(dicyclohexylphosphino)ferrocene, 2-(dicyclohexylphosphino)3,6-dimethoxy-2′,4′,6′-triisopropyl-1,1′-biphenyl, di(1-adamantyl)-n-butylphosphine, 1,2-ethanediylbis[dicyclohexyl]phosphine or a complex which is formed a palladium catalyst and a ligand such as [1,1′-bis(dicyclohexylphosphino) ferrocene]dichloropalladium(II). In one aspect, the catalytic system is palladium acetate and 1,1-bis(dicyclohexylphosphino)ferrocene. In another aspect, the catalytic system is [1,1′-bis(dicyclohexylphosphino) ferrocene]dichloropalladium(II).

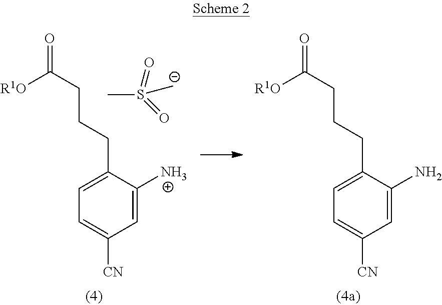

Scheme 2 illustrates the synthesis of compounds of formula (4a). Compounds of formula (4) can be treated with a base to provide free base (compounds of formula (4a)). Examples of bases used in this reaction include, but are not limited to, triethylamine, diisopropylethylamine; tributylamine, N-ethylpiperidine, and dimethylcyclohexylamine. In one aspect, the base is triethylamine.

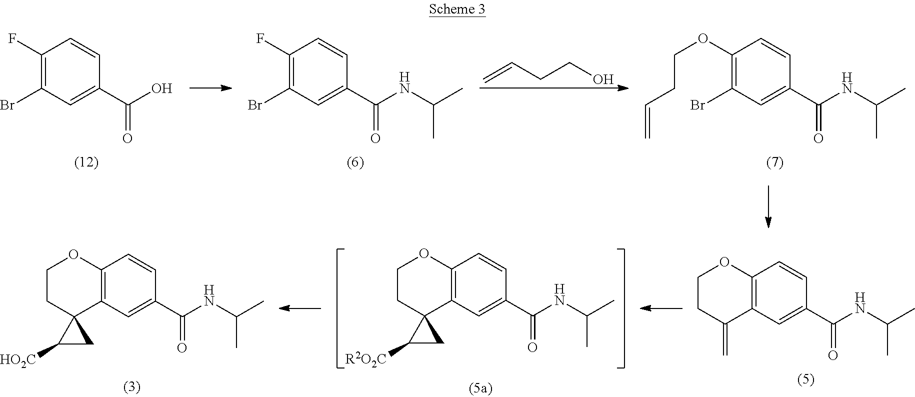

Scheme 3 depicts the preparation of Compound (3). Reaction of 3-fluoro-4-bromobenzoic acid (Compound (12)) with isopropylamine in the presence of a base and a coupling agent provides amide (Compound (6)). Examples of bases include, but are not limited to, 1-methyl-imidazole, 4,4-dimethylaminopyridine (DMAP), and diisopropylethylamine. In one aspect, the base is 1-methyl-imidazole. Representative coupling agents used in this reaction include, but are not limited to, N,N,N,N′-tetramethylchloroformamidinium hexafluorophosphate (TCFH), benzotriazol-1-yloxytris(dimethylamino)phosphonium hexafluorophosphate (BOP), benzotriazol-1-yl-oxytripyrrolidinophosphonium hexafluorophosphate (PyBOP), and 2-(1H-benzotriazol-1-yl)-1,1,3,3-tetramethyluronium hexafluorophosphate (HBTU). In one aspect, the coupling agent is N,N,N′,N′-tetramethylchloroformamidinium hexafluorophosphate (TCFH).

Treatment of amide (Compound (6)) with 3-buten-1-ol in the presence of a strong base provides Compound (7). Representative bases used in this reaction include, but are not limited to, potassium tert-butoxide, lithium tert-butoxide, sodium tert-butoxide, potassium hexamethyldisilazide, and lithium hexamethyldisilazide. In one aspect the base is potassium tert-butoxide. Cyclization of Compound (7) can be accomplished in the presence of a palladium catalytic system. Examples of palladium catalysts used in this reaction include, but are not limited to, palladium acetate and allyl palladium chloride dimer. In one aspect, the catalyst is palladium acetate. Representative ligands include, but are not limited to, 1,2-bis(diphenylphosphino)benzene, 2-dicyclohexylphosphino-2′,6′-di-isopropoxy-1,1′-biphenyl, (4-(N,N-dimethylamino)phenyl)di-tert-butyl phosphine, butyl di-1-adamantylphosphine, dicyclohexylphosphinodimethylaminobiphenyl, (±)-2,2′-bis(diphenylphosphino)-1,1′-binaphthalene, 1,1′-bis(di-t-butylphosphino)ferrocene, 2,2′-bis(dicyclohexylphosphino)biphenyl, tri-ortho-tolylphosphine, 2-(dicyclohexylphosphino)-2′,4′,6′-tri-i-propyl-1,1′-biphenyl, 1,1′-bis(dicyclohexylphosphino)ferrocene, 1,1′-bis(di-i-propylphosphino)ferrocene, 2-diphenylphosphino-2′,6′-bis(dimethylamino)-1,1′-biphenyl, dicyclohexyl(4-(N,N-dimethylamino)phenyl)phosphine, 2-(dicyclohexylphosphino)biphenyl, 2-(dicyclohexylphosphino)-2′-methylbiphenyl, tricyclohexylphosphonium tetrafluoroborate, 1,2-bis(diphenylphosphino)ethane, bis(2-dicyclohexylphosphinophenyl)ether, 2,2′-bis(diphenylphosphino)-1,1′-biphenyl, 1,3,5,7-tetramethyl-6-phenyl-2,4,8-trioxa-6-phosphaadamante, bis(2-diphenylphosphinophenyl)ether, triphenylphosphine, 2-(di-t-butylphosphino)-3,6-dimethoxy-2′,4′,6′-tri-i-propyl-1,1′-biphenyl, 2-(diphenylphosphino)-2′-(N,N-dimethylamino)biphenyl, [5-(diphenylphosphino)-9,9-dimethyl-9H-xanthen-4-yl](diphenyl)phosphine, 2-dicyclohexylphosphino-2′,6′-dimethoxy-1,1′-biphenyl, 2,2′-bis(diphenylphosphinomethyl)-1,1′-biphenyl, 1,4-bis(diphenylphosphino)butane, 9,9-dimethyl-4,5-bis(di-t-butylphosphino)xanthene, 2-(di-t-butylphosphino)biphenyl, tri-tert-butylphosphonium tetrafluoroborate, 2-di-t-butylphosphino-2′-methylbiphenyl, 2-dicyclohexylphosphino-2′,6′-bis(N,N-dimethylamino)biphenyl, 4,6-bis(diphenylphosphino)phenoxazine, tri-2-furylphosphine, 1,2-bis(dicyclohexylphosphino)ethane, 2-di-t-butylphosphino-2′-(N,N-dimethylamino)biphenyl, benzyldi-1-adamantylphosphine, 2-di-t-butylphosphino-2′,4′,6′-tri-i-propyl-1,1′-biphenyl, and dicyclohexyl[3-(dicyclohexylphosphino)propyl]phosphine. In one aspect, the ligand is selected from 1,2-bis(diphenylphosphino)benzene, 2-dicyclohexylphosphino-2′,6′-diisopropoxy-1,1′-biphenyl, (4-(N,N-dimethylamino)phenyl)di-tert-butyl phosphine, and butyl di-1-adamantylphosphine. In another aspect, the ligand is 1,2-bis(diphenylphosphino)benzene.

Alkene (Compound (5)) can be treated with a cyclopropanating agent in the presence of a catalyst and a chiral ligand to provide the chiral cyclopropane (Compound (5a)). Examples of cyclopropanating agents include, but are not limited to, C₁-C₆ alkyl diazoacetate and benzyl diazoacetate. Specific examples thereof include ethyl diazoacetate, methyl diazoacetate, n-butyl diazoacetate, benzyl diazoacetate, isopropyl diazoacetate, t-butyl diazoacetate. In one aspect, the cyclopropanating agent is ethyl diazoacetate. Representative catalysts used in this reaction include, but are not limited to, dichloro(p-cymene)ruthenium(II) dimer, dibromo(p-cymene)ruthenium(II) dimer, diiodo(p-cymene)ruthenium(II) dimer, tetrakisacetonitrile copper(I) triflate, copper(I) trifluoromethanesulfonate benzene complex, and bis(acetonitrile)dichloropalladium(II). In one aspect, the catalyst is selected from dichloro(p-cymene)ruthenium(II) dimer, dibromo(p-cymene)ruthenium(II) dimer, and diiodo(p-cymene)ruthenium(II) dimer. In another aspect, the catalyst is dichloro(p-cymene)ruthenium(II) dimer.

Chiral ligands that can be used in this reaction include, but are not limited to, from (S,S)-2,2′-(2,6-pyridinediyl)bis(4-isopropyl-2-oxazoline), (4S,4'S)-2,2′-(pentane-3,3′-diyl)bis(4-benzyl-4,5-dihydrooxazole), (R,R)-(+)-2,2′-isopropylidenebis(4-benzyl-2-oxazoline), 2,2′-bis[(4S)-4-benzyl-2-oxazoline], (3aS,3a'S,8aR,8a′R)-2,2′-(1,3-bis(3,5-di-t-butylphenyl)propane-2,2-diyl)bis(8,8a-dihydro-3aH-indeno[1,2-d]oxazole), [3aR-[2(3′aR*,8′aS*),3′aβ,8′aβ]]-(+)-2,2′-methylenebis[3a,8a-dihydro-8H-indeno[1,2-d]oxazole], (3aR,3′aR,8aS,8′aS)-2,2′-(1-methylethylidene)bis[3a,8a-dihydro-8H-indeno[1,2-d]oxazole], (−)-2,6-bis[(3aS,8aR)-3a,8a-dihydro-8H-indeno[1,2-d]oxazolin-2-yl]pyridine, (4S,4'S)-(−)-2,2′-(3-pentylidene)bis(4-isopropyloxazoline), 2,6-bis[(4R,5R)-4-methyl-5-phenyl-2-oxazolinyl]pyridine, (4S)-(+)-4-[4-(tert-butyl)phenyl]-α-[(4S)-4-[4-(tert-butyl)phenyl]-2-oxazolidinylidene]-2-oxazolineacetonitrile, (4S,4'S)-2,2′-(1-phenylpropane-2,2-diyl)bis(4-phenyl-4,5-dihydrooxazole), 2,2-bis(2-(4(S)-t-butyl-1,3-oxazolinyl))propane, 2,6-bis[(4R,5R)-4-methyl-5-phenyl-2-oxazolinyl]pyridine, (4S,4'S)-2,2′-(1,3-bis[4-(t-butyl)phenyl)propane-2,2-diyl]bis(4-phenyl-4,5-dihydrooxazole), 2,2′-methylenebis((4R,5S)-4,5-diphenyl-2-oxazoline), (S,S)-2,2′-methylenebis(4-phenyl-2-oxazoline), 2,2′-methylenebis[(4S)-4-tert-butyl-2-oxazoline], 2,2-bis(2-(4(S)-t-butyl-1,3-oxazolinyl))propane, 2,6-bis[(4R)-4-tert-butyloxazolin-2-yl]pyridine, (4S)-(+)-4-[4-(tert-butyl)phenyl]-α-[(4S)-4-[4-(tert-butyl)phenyl]-2-oxazolidinylidene]-2-oxazolineacetonitrile, (S)-4-(tert-butyl)-2-[5-(trifluoromethyl)pyridin-2-yl]-4,5-dihydrooxazole, (S)-4-tert-butyl-2-(2-pyridyl)oxazoline, and (R,R)-(−)-2,3-bis(tert-butylmethylphosphino)quinoxaline. In one aspect, the chiral ligand is selected from (S,S)-2,2′-(2,6-pyridinediyl)bis(4-isopropyl-2-oxazoline), (−)-2,6-bis[(3aS,8aR)-3a,8a-dihydro-8H-indeno[1,2-d]oxazolin-2-yl]pyridine, and 2,6-bis[(4R,5R)-4-methyl-5-phenyl-2-oxazolinyl]pyridine. In another aspect, the chiral ligand is (S,S)-2,2′-(2,6-pyridinediyl)bis(4-isopropyl-2-oxazoline).

Cyclopropyl ester (Compound of formula (5a)) can be hydrolyzed to provide the acid (Compound (3)) via treatment with a hydrolyzing agent. Representative hydrolyzing agents include, but are not limited to, sodium hydroxide, lithium hydroxide, potassium hydroxide, tetraethylammonium hydroxide, potassium trimethylsilanolate, tetramethylammonium hydroxide, tetra-n-propylammonium hydroxide, tetra-n-butylammonium hydroxide, and benzyltrimethylammonium hydroxide. In one aspect, the hydrolyzing agent is tetramethylammonium hydroxide. In another aspect, the hydrolyzing agent is sodium hydroxide.

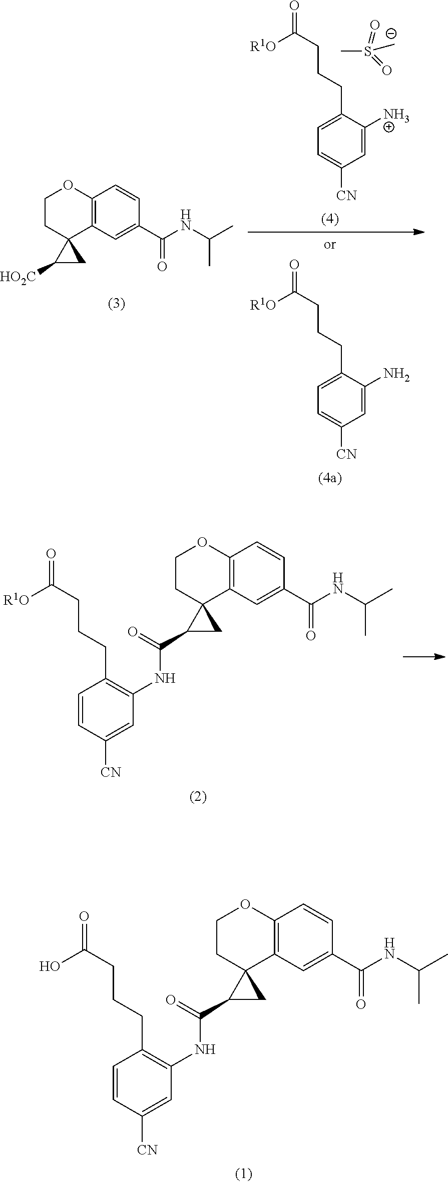

Scheme 4 shows the preparation of the compound (I). Compound (3) can be reacted with a compound of formula (4) or (4a) to provide amide (compound of formula (2)). Examples of bases include, but are not limited to, 1-methyl-imidazole, 4,4-dimethylaminopyridine (DMAP), and diisopropylethylamine. In one aspect, the base is 1-methyl-imidazole. In some aspects, the coupling is conducted in the presence of a coupling agent. Examples of coupling agents include, but are not limited to, N,N,N,N′-tetramethylchloroformamidinium hexafluorophosphate, 2,4,6-tripropyl-1,3,5,2,4,6-trioxatriphosphorinane-2,4,6-trioxide, diphenylphosphinic chloride, diphenyl phosphoryl chloride, N,N,N′,N′-tetramethylfluoroformamidinium hexafluorophosphate, N,N,N′,N′-tetramethyl-O-(1H-benzotriazol-1-yl)uronium hexafluorophosphate, benzotriazol-1-yl-oxytripyrrolidinophosphonium hexafluorophosphate, bis(2-oxo-3-oxazolidinyl)phosphinic chloride, 4-(4,6-dimethoxy-1,3,5-triazin-2-yl)-4-methylmorpholinium chloride, O-[(ethoxycarbonyl)cyanomethylenamino]-N,N,N′,N′-tetramethyluronium tetrafluoroborate, and 1-ethyl-3-(3-dimethylaminopropyl)carbodiimide, O—(N-succinimidyl)-N,N,N′,N′-tetramethyluronium hexafluorophosphate. In one aspect, the coupling agent is selected from N,N,N′,N′-tetramethylchloroformamidinium hexafluorophosphate, 2,4,6-tripropyl-1,3,5,2,4,6-trioxatriphosphorinane-2,4,6-trioxide, and diphenyl phosphoryl chloride. In another aspect, the coupling agent is N,N,N′,N′-tetramethylchloroformamidinium hexafluorophosphate. Hydrolysis of ester (Compound of formula (2)) provides the Compound (I). In one aspect, the hydrolysis is conducted in the presence of a hydrolyzing agent. Representative hydrolyzing agents include, but are not limited to, sodium hydroxide, lithium hydroxide, potassium hydroxide, tetramethylammonium hydroxide, cesium hydroxide, tetrabutylammonium hydroxide, tetrapropylammonium hydroxide, tetraethylammonium hydroxide, sodium trimethylsilanolate, potassium trimethylsilanolate, sodium carbonate, sodium bicarbonate, and postassium phosphate. In one aspect, the hydrolyzing agent is selected from sodium hydroxide, lithium hydroxide, and potassium hydroxide. In another aspect, the hydrolyzing agent is sodium hydroxide.

The compound (I) is converted into a salt using a known method. The salt is preferably a pharmaceutically acceptable salt. Preferably, the salt is water soluble. Examples of the pharmaceutically acceptable salt include acid addition salts, alkali metal salts, alkali-earth metal salts, ammonium salts, and amine salts. The acid addition salts may be inorganic acid salts, for example, such as hydrochloride, hydrobromate, hydroiodide, sulfates, phosphates, and nitrates, or organic acid salts, for example, such as acetates, lactates, tartrates, benzoates, citrates, methanesulfonate, ethanesulfonate, trifluoroacetate, benzenesulfonate, toluenesulfonate, isethionates, glucuronates, and gluconates. Examples of the alkali metal salts include potassium, and sodium. Examples of the alkali-earth metal salts include calcium, and magnesium. Examples of the ammonium salts include tetramethylammonium. Examples of the amine salts include triethylamine, methylamine, dimethylamine, cyclopentylamine, benzylamine, phenethylamine, piperidine, monoethanolamine, diethanolamine, tris(hydroxymethyl)aminomethane, lysine, arginine, and N-methyl-D-glucamine.

### Examples

The present invention is described below in detail by way of Examples, but the present invention is not limited by the following descriptions.

The compound names used in this specification are based on the computer program ACD/Name (registered trademark) or Chemdraw (registered trademark) Ultra, which generally generate chemical names according to IUPAC rules, or based on the IUPAC nomenclature.

Hereinafter, the Examples shown in Table 1 may be described the Compound number.

TABLE 1

| Compound No. | Name |
|---|---|
| Compound (2) | ethyl 4-[4-cyano-2-({(1′S,2′R)-6-[(propan-2- |
| | yl)carbamoyl]-2,3-dihydrospiro[[1]benzopyran-4,1′- |
| | cyclopropane]-2′-carbonyl}amino)phenyl]butanoate |
| | (may be written as ethyl 4-[4-cyano-2-({[(2′R,4S)- |
| | 6-(isopropylcarbamoyl)-2,3-dihydrospiro[chromene- |
| | 4,1′-cyclopropan]-2′- |
| | yl]carbonyl}amino)phenyl]butanoate) |
| Compound (3) | (1′S,2′R)-6-[(propan-2-yl)carbamoyl]-2,3- |
| | dihydrospiro[[1]benzopyran-4,1′-cyclopropane]-2′- |
| | carboxylic acid |
| | (may be written as (2′R,4S)-6- |
| | (isopropylcarbamoyl)-2,3-dihydrospiro[chromene- |
| | 4,1′-cyclopropan]-2′-yl]carboxylic acid) |
| Compound (4) | ethyl 4-(2-amino-4-cyanophenyl)butanoate |
| | monomethanesulfonate |
| Compound (4a) | ethyl 4-(2-amino-4-cyanophenyl)butanoate |
| Compound (5) | 4-methylidene-N-(propan-2-yl)-3,4-dihydro-2H-1- |
| | benzopyran-6-carboxamide |
| Compound (5a) | ethyl(1′S,2′R)-6-[(propan-2-yl)carbamoyl]-2,3- |
| | dihydrospiro[[1]benzopyran-4,1′-cyclopropane]-2′- |
| | carboxylate |
| | (may be written as ethyl (2′R,4S)-6- |
| | (isopropylcarbamoyl)-2,3-dihydrospiro[chromene- |
| | 4,1′-cyclopropan]-2′-yl]carboxylate) |
| Compound (6) | 3-bromo-4-fluoro-N-(propan-2-yl)benzamide |
| Compound (7) | 3-bromo-4-[(but-3-en-1-yl)oxy]-N-(propan-2- |
| | yl)benzamide |
| Compound (9) | ethyl but-3-enoate |

### 1. Preparation of Compound (4)

A vessel was charged with MTBE (15.0 L, 11.1 kg, 10 L/kg LR), absolute ethanol (0.86 kg, 1.3 equiv), triethylamine (2.20 kg, 1.5 equiv), and cooled to 0° C. under nitrogen. Crotonyl chloride (Compound (8), CAS No. 10487-71-5, 1.52 kg, 1 equiv, LR) was added while maintaining the batch temperature NMT 10′° C. The resulting mixture was agitated for NLT 2 h until residual starting material was NMT 1%. The mixture was filtered and the filter cake was washed with MTBE (2.24 kg, 3.0 L, 2.75 L/kg LR). The combined product rich filtrate solutions of Compound (9) were concentrated to 3 L/kg LR and tested for residual ethanol (NMT 1% v/v). 9-Borabicyclo[3.3.1]nonane (9-BBN) (20.2 kg, 0.5 M solution in THF, 1.55 equiv) was added to the reaction solution at 20′° C. The resulting mixture was aged at 20° C. for NLT 1 h until residual Compound (9) was NMT 1%. 3-Amino-4-chlorobenzonitrile (Compound (11), CAS No. 53312-79-1, 1.10 kg, 1.0 equiv, LR), 2-dicyclohexylphophino-2′,6′-dimethoxybiphenyl (SPhos) (0.030 kg, 0.010 equiv), palladium acetate (0.0082 kg, 0.0050 equiv), potassium phosphate (2.29 kg, 1.5 equiv), and water (0.18 kg, 1.5 equiv) were added to the vessel at 20° C. The mixture was heated to 60° C. until residual 3-amino-4-chlorobenzonitrile (Compound (11)) was NMT 1%. The mixture was cooled to 20° C. Water (6.7 L, 6.0 L/kg LR) and N-acetyl-L-cysteine (1.18 kg, 1.0 equiv) were added to the mixture followed by agitating at 20° C. for NLT 1 h. Agitation was stopped and the lower aqueous layer was removed. The rich organic phase was diluted with MTBE (8.17 kg, 11.0 L, 10.0 L/kg LR) and washed with water (5.5 L, 5 L/kg LR). The rich organic phase was concentrated to 4 L/kg LR and solvent exchange with ethanol until residual THF was NMT 0.2% v/v and residual water was NMT 0.8% w/w. Ethanol (6.8 kg, 8.6 L, 7.8 L/kg LR) was charged and the resulting mixture was agitated at 20° C. until a homogenous solution was obtained. Methanesulfonic acid (0.73 kg, 1.05 equiv) was charged over NLT 2 h at 20° C. followed by the addition of n-heptane (16.0 kg, 21.9 L, 20 L/kg LR) over NLT 2 h at 20° C. The resulting slurry was filtered, washed with a premixed solution of n-heptane/ethanol (6.6 L, 5:3 by volume, 3 L/kg LR), and dried in vacuo to afford Compound (4) as a solid (1.67 g, 71% yield). ¹H NMR (500 MHz, DMSO-d₆) δ 9.10-8.04 (br s, 3H), 7.25-7.16 (m, 3H), 4.05 (q, J=7.1 Hz, 2H), 2.61-2.52 (m, 2H), 2.44 (s, 3H), 2.34 (t, J=7.5 Hz, 2H), 1.77 (quin, J=7.6 Hz, 2H), 1.17 (t, J=7.1 Hz, 3H).

### 2. Alternative Preparation of Compound (4)

A vessel was charged with 2-methylbutanol (1.2 L, 12 L/kg LR), water (150 mL, 16 equiv), and ethyl 4-bromobutanoate (100 g, 1 equiv, LR) under nitrogen. Tris(dibenzylideneacetone)dipalladium(0) (2.42 g, 0.05 equiv), tricyclohexylphosphonium tetrafluoroborate (5.84 g, 0.03 equiv), bis(pinacolato)diboron (143 g, 1.1 equiv), and potassium phosphate (229 g, 2 equiv) were charged. The resulting mixture was heated to 65° C. for NLT 15 h. The mixture was cooled to 20° C. and the rich organic phase was washed with saturated aq ammonium chloride (1.0 L, 10 L/kg LR), diluted with toluene (0.50 L, 5 L/kg LR), and washed with water (1.0 L, 10 L/kg LR). The rich organic phase was diluted with MTBE (0.25 L, 2.5 L/kg) and washed with aq sodium chloride (0.10 L, 5 wt %, 1 L/kg). The rich organic phase was polish filtered and concentrated 2-3 L/kg LR, diluted with toluene (1.0 L, 10 L/kg LR), concentrated to 2-3 L/kg LR, and diluted with toluene (1.0 L, 10 L/kg LR). The concentrated solution was used as-is in the next step without further purification.

In a separate vial, anisole (5.0 mL, 4.0 L/kg LR), palladium (II) acetate (53.4 mg, 0.03 equiv), and 1,1-bis(dicyclohexlphosphino)ferrocene (148 mg, 0.033 equiv) were agitated at 20° C. for NLT 10 min followed by 3-amino-4-chlorobenzonitrile (1.15 g, 1 equiv, LR). Finally, the crude solution of the boronate (1.40 equiv) was charged to the vessel. Trisodium phosphate (1.61 g, 3 equiv) and water (6.9 mL, 6 L/kg LR) were charged followed by heating the mixture to 90° C. for NLT 20 h. The mixture was cooled to 20° C. and the lower aqueous phase was removed. The rich organic phase was washed with N-acetyl cysteine (1.15 g, 100 wt %) in water (11.5 mL, 10 L/kg LR), saturated aq sodium chloride (11.5 mL, 10 L/kg LR), and water (11.5 mL, 10 L/kg LR). The rich organic phase was concentrated to 1-2 L/kg LR and diluted with absolute ethanol (9.2 mL, 8 L/kg LR) and n-heptane (9.2 mL, 8 L/kg LR). Methanesulfonic acid (870 mg, 0.59 mL, 1.20 equiv) was charged at 20° C. followed by the addition of n-heptane (9.2 mL, 8 L/kg LR) over NLT 10 min at 20° C. The resulting slurry was filtered, washed with a premixed solution of n-heptane/ethanol (3.45 mL, 3:1 by volume, 3 L/kg LR), and dried in vacuo to afford Compound (4) as a solid (1.3 g, 55% yield).

### 3. Second Alternative Preparation of Compound (4)

A vessel was charged with 3-amino-4-chlorobenzonitrile (10.0 g, 1 equiv, LR), ethyl 4-(4,4,5,5-tetramethyl-1,3,2-dioxaborolan-2-yl)butanoate (22.2 g, 1.40 equiv), 2-methyltetrahydrofuran (66.7 mL, 6.67 L/kg LR) and N-methylpyrrolidone (13.3 mL, 1.33 L/kg LR) under nitrogen. The vessel was substituted with nitrogen atmosphere and then [1,1′-bis(dicyclohexylphosphino)ferrocene]dichloropalladium(II) (1.98 g, 0.04 equiv) was added. The vessel was substituted with nitrogen atmosphere and then degassed potassium carbonate solution (a mixture of potassium carbonate (31.7 g, 3.50 equiv) and water (30 mL, 3.0 L/kg LR)) were charged and then, the mixture was heated to 75° C. and stirred for NLT 14 h. The mixture was cooled to 40° C. and 14 wt % sodium chloride solution (60 mL, 6.0 L/kg LR) and N-acetyl cysteine (5.35 g, 0.500 equiv) were charged and the mixture was stirred at 40° C. for NLT 1 h. The aqueous phase was removed and water (80 mL, 8.0 L/kg LR), N-acetyl cysteine (5.35 g, 0.500 equiv) and potassium carbonate (9.06 g, 1.00 equiv) were charged to the organic phase and the mixture was stirred at 40° C. for NLT 1 h. The aqueous phase was removed and water (80 mL, 8.0 L/kg LR), N-acetyl cysteine (5.35 g, 0.500 equiv) and potassium carbonate (9.06 g, 1.00 equiv) were charged to the organic phase and the mixture was stirred at 40° C. for NLT 1 h. The aqueous phase was removed and the rich organic phase was washed with 10 wt % sodium chloride solution (60 mL, 6.0 L/kg LR). The rich organic phase was filtered through a pad of celite and the celite pad was washed with 2-methyltetrahydrofuran (40 mL, 4.0 L/kg LR). The filtrate was concentrated to 4 L/kg LR. Two put and takes of ethanol (110 mL, 11.0 L/kg LR) were completed with an end point of 4 L/kg LR. Ethanol (80 mL, 8.0 L/kg LR) was added and the mixture was heated to 35° C. Methanesulfonic acid (3.15 g, 0.500 equiv) was charged at 35° C. and then the mixture was cooled to 20° C. over 1 h. Methanesulfonic acid (4.41 g, 0.700 equiv) was charged over 1 h and then n-heptane (200 mL, 20 L/kg LR) was added over 2 h at 20° C. The resulting slurry was filtered, washed with a premixed solution of n-heptane/ethanol (99 mL, 5:3 by volume, 9.9 L/kg LR), and dried in vacuo to afford Compound (4) as a solid (16.6 g, 77% yield).

### 4. Preparation of Compound (4a)

A vessel was charged with Compound (4) (15.0 g, 45.7 mmol), aged for about 5 minutes, charged with triethylamine (5.55 g, 54.8 mmol), agitated for not less than 1 hour; charged with water (225 mL) over NLT 3 hours, and aged overnight. The precipitate was collected by filtration, rinsed with 3:1 water/ethanol (45 mL), washed with water (30 mL), and dried under vacuum at 50° C. to provide Compound (4a) (9.74 g, 41.9 mmol, 91.8% yield).

### 5. Alternative Preparation of Compound (4a)

A vessel was charged with Compound (4) (10.0 g, 1 equiv, LR), acetonitrile (10 mL, 1.0 L/kg LR) and ethanol (30 mL, 3.0 L/kg LR). Triethylamine (4.01 g, 1.3 equiv) was charged and then water (60 mL, 6.0 L/kg LR) was added at 25° C. over 3 h. The mixture was cooled to 0° C. and aged for NLT 1 h. The resulting slurry was filtered, washed with a premixed solution of water/ethanol (30 mL, 2:1 by volume, 3.0 L/kg LR), and dried in vacuo to afford Compound (4a) as a solid (6.53 g, 92% yield).

### 6. Preparation of Compound (6)

A vessel was charged with acetonitrile (11.86 kg, 5 L/kg LR), 1-methyl-imidazole (2.46 kg, 2.2 equiv), 3-fluoro-4-bromobenzoic acid (Compound (12), CAS No. 153556-42-4, 3.0 kg, 1 equiv, LR), and cooled to 0° C. Isopropylamine (0.90 kg, 1.1 equiv) was added while maintaining the batch temperature NMT 10° C. N,N,N′,N′-tetramethylchloroformamidinium hexafluorophosphate (4.23 kg, 1.1 equiv) was added while maintaining the batch temperature below 20° C. The mixture was warmed to 20° C. and aged for NLT 1 h until residual starting material was NMT 1%. The mixture was concentrated to 4 L/kg and warmed to 50° C. Water (36 kg, 12 L/kg) was added over NMT 4 h while maintain the batch temperature at 50° C. Upon completion of the water addition, the mixture was cooled to 20° C. over NLT 1 h followed by aging at 20° C. for NLT 2 h. The slurry was filtered, washed with a premixed solution of acetonitrile/water (3 L, 1:4 by volume, 3 L/kg LR), and dried in vacuo to afford Compound (6) as a solid (3.3 kg, 94% yield). ¹H NMR (DMSO-d₆, 500 MHz): δ=8.35 (br d, J=6.7 Hz, 1H), 8.19 (br d, J=5.5 Hz, 1H), 7.89-7.93 (m, 1H), 7.46 (br t, J=8.7 Hz, 1H), 4.07 (dq, J=13.2, 6.7 Hz, 1H), 1.15 ppm (br d, J=6.7 Hz, 6H).

### 7. Preparation of Compound (7)

A vessel was charged with THF (670 g, 3 L/kg LR), 3-buten-1-ol (141 g, 2 equiv), and Compound (6) (250 g, 1 equiv, LR). The mixture was agitated until all solids dissolved. Potassium tert-butoxide (755 g, 20 wt % solution in THF, 1.4 equiv) was added while maintaining the temperature NMT 25° C. Upon completion of addition, the mixture was aged NLT 16 h at 20° C. until residual starting material was NMT 1%. The mixture was washed with aq potassium bicarbonate (1.25 L, 5 wt %, 5 L/kg LR). The rich organic phase was warmed to 50° C., solvent exchanged into acetonitrile (NMT 5% residual THF), and adjusted to a volume of 5 L/kg. Water (2.5 L, 10 L/kg LR) was added over NLT 2 h while maintaining the batch temperature at 50° C. Upon completion of the water addition, the mixture was cooled to 20° C. over NLT 1 h followed by aging at 20° C. for NLT 2 h. The slurry was filtered, washed with a premixed solution of acetonitrile/water (750 mL, 1:2 by volume, 3 L/kg LR), and dried in vacuo to afford Compound (7) as a solid (277 g, 92% yield). ¹H NMR (DMSO-d₆, 500 MHz): δ=8.18 (br d, J=7.6 Hz, 1H), 8.09 (d, J=2.1 Hz, 1H), 7.85 (dd, J=8.5, 2.1 Hz, 1H), 7.17 (d, J=8.9 Hz, 1H), 5.88-5.92 (m, 1H), 5.19 (dd, J=17.2, 1.7 Hz, 1H), 5.10 (d, J=10.4 Hz, 1H), 4.16 (t, J=6.6 Hz, 2H), 4.07 (dd, J=13.9, 6.9 Hz, 1H), 2.51-2.54 (m, 2H), 1.15 ppm (d, J=6.4 Hz, 6H).

### 8. Alternative Preparation of Compound (7)

A vessel was charged with THF (60 mL, 2.0 L/kg LR), 3-buten-1-ol (16.6 g, 2.00 equiv), and Compound (6) (30 g, 1 equiv, LR). The mixture was agitated until all solids dissolved. Potassium tert-butoxide (150 g, 1.0 mol/L solution in THF, 1.50 equiv) was added while maintaining the temperature NMT 25° C. Upon completion of addition, the mixture was aged NLT 16 h at 20° C. until residual starting material was NMT 1%. A portion of the reaction mixture (116 g, corresponding to 13 g of Compound (6)) was charged with MTBE (78 mL, 6.0 L/kg LR) and the mixture was washed with 5 wt % potassium hydrogen carbonate solution (65 mL, 5 L/kg LR). The rich organic phase was concentrated to 4 L/kg LR. Two put and takes of acetonitrile (65 mL, 5.0 L/kg LR) were completed with an end point of 5 L/kg LR. The resulting mixture was cooled to 20° C. and the precipitation of Compound (7) was confirmed. Water (130 mL, 10 L/kg LR) was added over NLT 4 h and the mixture was aged at 20° C. for NLT 0.5 h. The slurry was filtered, washed with a premixed solution of acetonitrile/water (39 mL, 1:2 by volume, 3.0 L/kg LR), and dried in vacuo to afford Compound (7) as a solid (14.0 g, 90% yield).

### 9. Preparation of Compound (5)

To a 100 mL round-bottom flask with stir bar was added 2-methyltetrahydrofuran (22.5 mL, 4.5 L/kg (vol)) and isopropanol (2.5 mL, 0.5 L/kg (vol)). The flask was sealed with a septa and degassed by sparging with nitrogen for 20 minutes. Compound (7) (4.99 g, 1.0 equiv, LR) was then charged under nitrogen followed by 1,2-bis(diphenylphosphino)benzene (130.2 mg, 0.018 equiv) and palladium(II) acetate (35.3 mg, 0.01 equiv) under nitrogen. After stirring for 10 min, tetramethylammonium acetate (4.3 g) was charged under nitrogen. The stream was degassed by sparging with nitrogen for 10 minutes and then heated to 75° C. overnight. After 15.5 h, an aliquot was taken and subjected to UPLC analysis, which showed complete conversion. The stream was diluted with MeTHF (10 L/kg (vol), 50 mL), and cooled to room temperature. The stream was filtered and the solids rinsed with MeTHF (2 L/kg (vol), 10 mL). The filtrate solvent was swapped to isopropanol by distillation. The stream was distilled to 5 vol. Three put and takes of isopropanol (5 L/kg (vol)) were completed with an end point of 5 vol for the first two puts and 7 vol for the third. The stream was then transferred to a 250 mL RBF and heated to 50° C. Water (14 L/kg (vol), 140 mL) was added over 2 h during which crystallization occurred, cooled to 20° C., and aged at room temperature for 2 h. The product was isolated by filtration, and the cake was washed with 1:2 isopropanol/water (4 L/kg (vol), 20 mL). The solids were then dried at 50° C. overnight with full vacuum and nitrogen sweep. After drying, 3.21 g of a white solid were obtained. HPLC and ¹H NMR confirmed formation of Compound (5) in 87% yield.

### 10. Alternative Preparation of Compound (5)

A vessel was charged with N-methylpyrrolidone (75 mL, 5 L/kg LR), which was then degassed, followed by 1, 2-bis(diphenylphosphino)benzene (0.267 g, 0.012 equiv), and palladium (II) acetate (0.108 g, 0.01 equiv) under nitrogen. The resulting mixture was aged at 20° C. under nitrogen for NLT 0.5 h. Compound (7) (15 g, 1 equiv, LR) and tetramethylammonium acetate (12.85 g, 2.0 equiv) were charged followed by heating the mixture to 75° C. for NLT 12 h. When residual starting material was NMT 1%, the mixture was cooled to 50° C. Acetone (45 mL, 3 L/kg LR) was charged while maintaining the batch temperature at 50° C. Water (90 mL, 6 L/kg LR) was charged while maintaining the batch temperature at 50° C. Compound (5) seed crystals (0.15 g, 1%) were charged (though not required) and the mixture was aged at 50° C. for NLT 0.5 h. Water (90 mL, 6 L/kg LR) was added over NLT 4 h while maintaining the batch temperature at 50° C. The mixture was cooled to 20° C. and aged for NLT 2 h. The slurry was filtered, washed with a premixed solution of acetone/water (120 mL, 1:4 by volume, 8 L/kg LR), and dried in vacuo to afford Compound (5) as a solid (9.81 g, 88% yield). ¹H NMR (600 MHz, DMSO-d₆) δ 8.17 (d, J=2.1 Hz, 1H), 8.12 (d, J=7.6 Hz, 1H), 7.69 (dd, J=8.5, 2.1 Hz, 1H), 6.86 (d, J=8.5 Hz, 1H), 5.70 (s, 1H), 5.02 (s, 1H), 4.22 (t, J=5.7 Hz, 2H), 4.10 (m, 1H), 2.65 (t, J=5.5 Hz, 2H), 1.16 (d, J=6.7 Hz, 6H).

### 11. Second Alternative Preparation of Compound (5)

A vessel was charged with 2-methyltetrahydrofuran (100 mL, 5 mL/g LR), Compound (6) (20 g, 1 equiv, LR), 3-buten-1-ol (12.2 g, 2.2 equiv), and sodium tert-butoxide (12.18 g, 1.6 equiv) at 25° C. under nitrogen. The resulting mixture was heated to 40° C. for NLT 6 h until residual starting material was NMT 1%. The mixture was diluted with 2-methyltetrahydrofuran (80 mL, 4 mL/g LR) and washed with aq sodium carbonate (100 mL, 5 mL/g LR) at 40° C. The rich organic phase was washed with aq sodium chloride (100 mL, 13 wt %, 5 mL/g LR) and azeotropically dried at constant volume until residual water was NMT 0.4 wt %. 1,2-bis(diphenylphosphino)benzene (460 mg, 0.013 equiv), palladium (II) acetate (172 mg, 0.01 equiv), and tetramethylammonium acetate (20.43 g, 2 equiv) were charged to the reaction followed by heating to 75° C. for NLT 16 h and residual Compound (7) was NMT 1%. The mixture was cooled to 50° C. and washed with water (100 mL, 5 mL/g LR). The rich organic phase was azeotropically dried at constant volume until residual water was NMT 0.5 wt %. The rich organic phase was polish filtered at 50° C., concentrated to 5 mL/g LR, and held at 40° C. for NLT 1 h (a seed bed formed). n-Heptane (200 mL, 10 L/kg LR) was charged over NLT 2 h followed by cooling to 20° C. and aging at 20° C. for NLT 2 h. The resulting slurry was filtered, washed with a premixed solution of 2-methyltetrahydrofuran/n-heptane (40 mL, 2:3 by volume, 2 L/kg LR) followed by n-heptane (40 mL, 2 L/kg LR), and dried in vacuo to afford Compound (5) as a solid (14.2 g, 79% yield).

### 12. Third Alternative Preparation of Compound (5)

A vessel was charged with N,N-dimethylacetamide (350 mL, 3.5 L/kg LR), tripotassium phosphate (136 g, 2.0 equiv), palladium acetate (1.44 g, 0.0200 equiv) and 1,2-bis(diphenylphosphino)benzene (4.29 g, 0.0300 equiv). The vessel was substituted with nitrogen atmosphere and the mixture was heated to 85° C. over 2 h and stirred at 85° C. for 1 h. The vessel was charged with Compound (7) (100 g, 1 equiv, LR) in N,N-dimethylacetamide (150 mL, 1.5 L/kg LR) at 85° C. over 4 h and then the mixture was stirred for overnight. After cooling the reaction mixture to 50° C., ethyl acetate (1.0 L, 10 L/kg LR), 20 wt % sodium chloride solution (1.0 L, 10 L/kg LR) and N-acetyl cysteine (26.1 g, 0.500 equiv) were charged and the mixture was stirred at 50° C. for NLT 1 h. The aqueous phase was removed and the rich organic phase was charged with 20 wt % sodium chloride solution (1.0 L, 10 L/kg LR), N-acetyl cysteine (26.1 g, 0.500 equiv) and potassium carbonate (44.2 g, 1.00 equiv) and stirred at 50° C. for NLT 1 h. The aqueous phase was removed and the rich organic phase was charged with 20 wt % sodium chloride solution (1.0 L, 10 L/kg LR) and potassium carbonate (44.2 g, 1.00 equiv) and stirred at 50° C. for NLT 15 min. The aqueous phase was removed and the rich organic phase was washed with 20 wt % sodium chloride solution (1.0 L, 10 L/kg LR) at 50° C. The organic phase was concentrated to 5 L/kg LR and three put and takes of isopropanol (5 L/kg LR) were completed with an end point of 5 L/kg LR for the first two puts and 6 L/kg LR for the third. The resulting mixture was heated to 50° C. Water (300 mL, 3.0 L/kg LR) was added over 15 minutes at 50° C. during which crystallization occurred. Additional water (1.1 L, 11 L/kg LR) was added over 3 h at 50° C., and then the mixture was cooled to 20° C., and aged for 2 h. The resulting slurry was filtered, washed with a premixed solution of isopropanol/water (900 mL, 1:2 by volume, 9 L/kg LR), and dried in vacuo to afford Compound (5) as a solid (66.8 g, 90% yield).

### 13. Preparation of Compound (3)

A vessel was charged with 2-methyltetrahydrofuran (1.5 L), Compound (5) (100 g, 432.3 mmol), (S,S)-(2,6-bis(4-isopropyl)-2-oxazolin-2-yl)pyridine (3.949 g, 12.97 mmol), and additional 2-methyltetrahydrofuran (0.5 mL) was purged with nitrogen for about 10 minutes and charged with dichloro(p-cymene)ruthenium(II) dimer (4.094 g, 6.485 mmol), and heated to 50° C. Ethyldiazoacetate (EDA, prepared as described in Maurya, R. A. et al., Green Chem., 2014, 16, p. 116, 0.44 L, 15 wt % solution in toluene, 1.2 equiv) was charged over 115 minutes and the reaction was aged overnight at 50° C. Based on HPLC analysis, the reaction was not completed, so additional 0.2 equiv of EDA was added over 20 minutes. After 2 hours at 50° C. the reaction was complete by HPLC analysis. After 24 hours the mixture was charged with tetramethylammonium hydroxide (465.5 mL, 1297 mmol) and aged overnight at 50° C. The mixture was cooled to 20° C. and the organic phase was washed with water (200 mL). The aqueous phase was combined with the earlier aqueous phase, aged at 20° C. for 72 hours, heated to 45° C., and charged with acetone (967 mL), water (193 mL), and HCl (37%) in water (107 mL). The batch was aged for 1.5 hours at 45° C. The mixture was cooled to 35° C. over 2 hours, aged 1 hour, cooled to 20° C. over 1 hour, and aged 1 hour. The mixture was filtered and washed with water (540 mL) and acetone (60 mL), then dried under vacuum at 50° C. overnight to provide Compound (3) (68.5 g, 54.8% yield).

### 14. Alternative Preparation of Compound (3)

A vessel was charged with 2-methyltetrahydrofuran (1.5 L, 15 mL/g LR), Compound (5) (0.1 kg. 1 equiv, LR), (S,S)-(2,6-bis(4-isopropyl)-2-oxazolin-2-yl)pyridine ((S,S)-iPr-Pybox) (3.95 g, 0.03 equiv), and [RuCl₂(cymene)]₂ (4.09 g, 0.015 equiv) under nitrogen. Additional 2-methyltetrahydrofuran (0.5 L, 5 mL/g LR) was charged. The mixture was heated to 50° C. for NLT 1 h. Ethyldiazoacetate (prepared as described in Maurya, R. A. et al., Green Chem., 2014, 16, p. 116, 0.44 L, 15 wt % solution in toluene, 1.20 equiv) was charged over 3 h while maintaining batch temperature below 60° C. Upon completion of addition, the mixture was aged at 50° C. until residual starting material was NMT 2%. Tetramethylammonium hydroxide (0.466 L, 25% w/w in water, 3.0 equiv) was added to the rich organic stream containing Compound (5a) while maintaining the batch temperature below 70° C. Upon completion of the addition, the mixture was aged at 50° C. for NLT 12 h until the concentration of Compound (5a) in the organic phase was NMT 1.1 mg/mL. The mixture was cooled to 15° C. and the lower, product rich aqueous layer was removed. The lean organic phase was extracted with water (0.2 L, 2 mL/g LR). The product rich aqueous phase was removed. All product rich aqueous phases were combined, diluted with water (0.2 L, 2 mL/g LR) and acetone (1 L, 10 mL/g LR), and heated to 50° C. Aqueous hydrochloric acid (0.12 L, 12 M, 3.1 equiv) was added to the mixture at 50° C. Compound (3) seeds (1.0 g, 0.01 g/g LR) were added to the mixture and aged for NLT 2 h at 50° C. The mixture was cooled to 20° C. over NLT 8 h followed by aging at 20° C. for NLT 2 h. The slurry was filtered, washed with a premixed solution of water/acetone (0.6 L, 9:1 by volume, 6 mL/g LR), and dried in vacuo. The crude solid was recrystallized from acetone/water to afford Compound (3) as a solid (68.7 g, 54.9% yield). ¹H NMR (600 MHz, DMSO-d₆) δ 8.02 (d, J=7.7 Hz, 1H), 7.61 (br d, J=8.5 Hz, 1H), 7.29 (br s, 1H), 6.81 (d, J=8.5 Hz, 1H), 4.29 (m, 1H), 4.10 (m, 1H), 4.08 (m, 1H), 2.18 (t, J=7.3 Hz, 1H), 2.08 (br t, J=5.0 Hz, 2H), 1.70 (dd, J=8.2, 4.9 Hz, 1H), 1.43 (t, J=5.7 Hz, 1H), 1.15 (d, J=6.4 Hz, 6H).

### 15. Second Alternative Preparation of Compound (3)

A vessel was charged with toluene (70 mL, 7.0 L/kg LR), Compound (5) (10 g, 1 equiv, LR), (S,S)-(2,6-bis(4-isopropyl)-2-oxazolin-2-yl)pyridine ((S,S)-iPr-Pybox) (182 mg, 0.014 equiv), and [RuCl₂(cymene)]₂ (185 mg, 0.007 equiv) under nitrogen. Additional toluene (5 mL, 0.5 L/kg LR) was charged. The mixture was heated to 55° C. and stirred for NLT 1 h. Ethyl diazoacetate (15 wt % solution in toluene, 1.05 equiv) was charged at 55° C. over 3 h. Upon completion of addition, the line was washed with toluene (5 mL, 0.5 L/kg LR) and the mixture was aged at 55° C. for 1 h. Additional ethyl diazoacetate (0.2 equiv) was charged over 0.5 h and the mixture was stirred at 55° C. for 1 h. The reaction mixture was cooled to 45° C. and then charged with ethanol (20 mL, 2 L/kg LR) and 4 mol/L sodium hydroxide solution (32.4 mL, 3.0 equiv). The mixture was stirred at 45° C. for NLT 8 h. The mixture was cooled to 25° C. and water (17.5 mL, 1.75 L/kg LR) was added. After stirring the mixture for 0.5 h, the organic phase was removed. The product rich aqueous layer was charged with tetrahydrofuran (75.5 mL, 7.55 L/kg LR), citric acid solution (citric acid monohydrate (8.62 g, 0.95 equiv) and water (6.9 mL, 0.69 L/kg LR)). Hydrochloric acid solution (35% hydrochloric acid (7.2 g) and water (3.3 mL)) was added and the mixture was stirred at 25° C. for 0.5 h. The aqueous phase was removed and the total content of Compound (3) and its enantiomer in the organic phase was analyzed by HPLC. The rich organic phase was concentrated to 9 L/kg LR and tetrahydrofuran was added to adjust tetrahydrofuran/[Compound (3) and its enantiomer] ratio to 3.8/1 (g/g). The mixture was heated to 45° C. and then ethanol (20 mL, 2 L/kg LR) was charged. The mixture was cooled to 25° C. over 1 h followed by the addition of Compound (3) seeds (10 mg, 0.001 g/g LR). Water (80 mL, 8 L/kg LR) was charged over 3 h and the mixture was stirred at 25° C. for 1 h and then cooled to 18° C. After stirring the mixture for overnight, the resulting slurry was filtered, washed with a premixed solution of ethanol/water (24 mL, 5:3 by volume, 2.4 L/kg LR), and dried in vacuo to afford Compound (3) as a solid (7.41 g, 59% yield).

### 16. Preparation of Compound (2)

A vessel was charged with N,N,N′,N′-tetramethylchloroformamidinium hexafluorophosphate (72.9 g, 1.26 equiv) and acetonitrile (250 mL, 4.17 mL/g LR). The mixture was cooled to 0° C. 1-Methylimidazole (64.8 g, 3.82 equiv) was added dropwise over ˜5 min. The flask was rinsed with acetonitrile (60 mL, 1 mL/g LR). Compound (3) (59.8 g, 1 equiv, LR) was added to the flask. The flask was rinsed with acetonitrile (50 mL, 0.83 mug LR). Compound (4) (72.7 g, 1.07 equiv) was charged. The flask was rinsed with tetrahydrofuran (150 mL, 2.5 mL/g LR). The reaction temperature was increased to 50° C. and aged until residual starting material was NMT 1%. Water (585 mL, 9.75 mL/g LR) was added over NLT 2 h followed by cooling the mixture from 50° C. to 20° C. over NLT 2 h. The resulting slurry was filtered and washed with a premixed solution of water/acetonitrile (540 mL, 1:1 by volume, 9 mL/g LR). The wet cake was dried in vacuo to afford Compound (2) (100.4 g, 96% yield). ¹H NMR (600 MHz, DMSO-d₆) δ 9.87 (s, 1H), 8.05 (d, J=7.7 Hz, 1H), 7.93 (br s, 1H), 7.65 (dd, J=8.5, 1.8 Hz, 1H), 7.56 (dd, J=8.1, 1.3 Hz, 1H), 7.43 (br s, 1H), 7.42 (d, J=8.1 Hz, 1H), 6.83 (d, J=8.5 Hz, 1H), 4.32 (m, 1H), 4.10 (m, 1H), 4.08 (m, 1H), 3.97 (q, J=7.2, 4.1 Hz, 2H), 2.66 (m, 2H), 2.49 (m, 1H), 2.28 (t, J=7.3 Hz, 2H), 2.14 (m, 2H), 1.81 (dd, J=8.2, 4.9 Hz, 1H), 1.75 (m, 2H), 1.60 (t, J=5.7 Hz, 1H), 1.17 (d, J=6.6 Hz, 6H), 1.14 (t, J=7.2 Hz, 3H).

### 17. Preparation of Compound (2) Using Compound (4a)

A vessel was charged with acetonitrile (30.95 g), treated with N,N,N′,N′-tetramethylchloroformamidinium hexafluorophosphate (6.03 g, 21.3 mmol), treated with Compound (3) (5.00 g, 17.3 mmol) followed by Compound (4a) (4.60 g, 19.8 mmol) and then charged with THF (15.50 g) and 1-methylimidazole (4.24 g, 51.6 mmol). The mixture was heated to 50° C. and then cooled to room temperature when reaction completion in-process control passed. The mixture was heated to 30° C. and treated with 17.5 mL water in 5 mL aliquots slowly. Another 32.5 mL of water was added and the reaction was held at 20° C. for 72 hours. The precipitate was collected by filtration and washed with a 1:1 mixture of acetonitrile and water (9 L/kg) and then dried in vacuum at 50° C. to provide 7.52 g (86% yield) of Compound (2).

### 18. Alternative Preparation of Compound (2) Using Compound (4a)

A vessel was charged with acetonitrile (30 mL, 6 L/kg LR), THF (12.5 mL, 2.50 L/kg LR), Compound (3) (5.0 g, 1 equiv, LR), Compound (4a) (4.3 g, 1.07 equiv) and N,N,N′,N′-tetramethylchloroformamidinium hexafluorophosphate (5.92 g, 1.22 equiv). 1-Methylimidazole (3.87 g, 2.73 equiv) was added while maintaining the batch temperature below 30° C. The mixture was heated to 55° C. and stirred for 3 h, and then cooled to 45° C. Compound (2) seeds (5 mg, 0.001 g/g LR) was added and the mixture was stirred at 45° C. for NLT 15 minutes. Water (50 mL, 10 L/kg LR) was added at 45° C. over 2 h and then the mixture was cooled to room temperature. After stirring the mixture for overnight, the resulting slurry was filtered, washed with a premixed solution of acetonitrile/water (45 mL, 1:1 by volume, 9 L/kg LR), and dried in vacuo to afford Compound (2) as a solid (8.06 g, 93% yield).

### 19. Preparation of Compound (I)

A vessel with an overhead stirrer was charged with Compound (2) (8.0 g, 15.9 mmol) and THF (35 mL) at 15-20° C. Water (3.52 mL) was charged followed by aq sodium hydroxide (50 wt % in water, 1.27 mL, 23.8 mmol) and purged with nitrogen. The resulting mixture was stirred at 20° C. overnight, then charged with additional water (7.2 mL, 400 mmol) and allowed to stir at room temperature for 24 hours. The mixture was charged with HCl (2.0 mL, 24 mmol) at room temperature and stirred for NLT 1 hour. Acetone (6.60 mL, 90 mmol) was added and then continuously charged (total addition 48 mL) until a homogenous mixture was obtained. Water (52 mL) was added over 5-10 minutes and stirred at room temperature overnight. The precipitate was collected by filtration, washed with a pre-mixed solution of acetone (2 L/kg (vol)) and water (3 L/kg (vol)), then dried under vacuum at 65° C. over 3 days to provide 6.38 g (85% yield) of Compound (I).

### 20. Alternative Preparation of Compound (I)

A vessel was charged with Compound (2) (16.0 g, 1.0 equiv, LR) and THF (96 mL, 6 mL/g LR) at 15-20° C. Water (17.6 mL, 1.1 mL/g LR) was charged followed by aq sodium hydroxide (9.5 mL, 5 M, 1.5 equiv). The resulting mixture was agitated at 15-20° C. until residual starting material was NMT 0.5%. Aq hydrochloric acid (9.5 mL, 6 M, 1.8 equiv) was added and the resulting mixture was agitated for 30 min. Agitation was stopped, the phases were allowed to split, and the lower aqueous layer was removed. The product rich organic phase was polish filtered. The filtrate was diluted with acetone (53 mL, 3.3 mL/g LR) and water (64 mL, 4.0 mL/g LR). Seed crystals (0.32 g, 0.02 g/g LR) were added followed by wet milling for NLT 4 h and the resulting slurry was aged at 20° C. for NLT 12 h. Water (104 mL, 6.5 mL/g LR) was charged over NLT 5 h followed by aging the slurry for NLT 1 h. The slurry was filtered, the wet cake was washed with water/acetone (80 mL, 3:2 by volume, 5 mL/g LR), and the cake was dried in vacuo to afford Compound (I) (13.1 g, 87% yield). ¹H NMR (600 MHz, DMSO-d₆): δ 1.153 (d, J=6.6 Hz, 3H), 1.151 (d, J=6.6 Hz, 3H), 1.56-1.60 (m, 1H), 1.69-1.76 (m, 2H), 1.78 (dd, J=7.8, 4.8 Hz, 1H), 2.06-2.16 (m, 2H), 2.20 (t, J=7.5 Hz, 2H), 2.44-2.49 (m, 1H), 2.60-2.70 (m, 2H), 4.04-4.13 (m, 2H), 4.28-4.34 (m, 1H), 6.83 (d, J=8.6 Hz, 1H), 7.40 (d, J=7.8 Hz, 1H), 7.42 (d, J=1.8 Hz, 1H), 7.56 (dd, J=7.8, 1.5 Hz, 1H), 7.64 (dd, J=8.6, 2.1 Hz, 1H), 7.87 (d, J=1.5 Hz, 1H), 8.04 (d, J=7.8 Hz, 1H), 9.90 (s, 1H), 12.09 (s, br, 1H).

### 21. Second Alternative Preparation of Compound (I)

A vessel was charged with Compound (2) (20.0 g, 1.0 equiv, LR) and THF (120 mL, 6.0 mL/g LR) at 15-20° C. Water (22.0 mL, 1.1 mL/g LR) was charged followed by the addition of sodium hydroxide solution (11.9 mL, 5 mol/L, 1.5 equiv). The resulting mixture was agitated at 20-25° C. until residual starting material was NMT 0.2%. Hydrochloric acid solution (9.80 mL, 6 mol/L, 1.48 equiv) was added and then pH of the aqueous phase was adjusted to 3.0-4.0 with sodium hydroxide solution (1 mol/L) and hydrochloric acid solution (1 mol/L). The aqueous phase was removed and the product rich organic phase was polish filtered. The filtrate was diluted with acetone (100 mL, 5.0 mL/g LR) and water (74 mL, 3.7 mL/g LR). Seed crystals (0.40 g, 0.02 g/g LR) were added and then water (160 mL, 8.0 L/kg LR) was added at 22° C. over 6 h. The resulting slurry was cooled to 0° C. and stirred for overnight. The slurry was filtered and the wet cake was washed with a premixed solution of water/acetone (100 mL, 3:2 by volume, 5.0 mL/g LR), and the cake was dried in vacuo to afford the Compound (I) (17.9 g, 95% yield).

The present method is both efficient and cost-effective method that is reduced the number of steps, improved the stereoselectivity of cyclopropanation and changed a step of exomethylene moiety formation by Witting reaction to a step of exomethylene moiety formation by Heck reaction. For example, in example 12, the compound (5) was obtained in a high yield of 90% by the step of exomethylene moiety formation by Heck reaction. For example, in examples 13-15, the diastereoselectivity of compound (3) was improved at {[ethyl (1'S,2′R)-6-[(propan-2-yl)carbamoyl]-2,3-dihydrospiro[[1]benzopyran-4,1′-cyclopropane]-2′-carboxylate]+[ethyl (1′R,2'S)-6-[(propan-2-yl)carbamoyl]-2,3-dihydrospiro[[1]benzopyran-4,1′-cyclopropane]-2′-carboxylate])/{[ethyl (1'S,2'S)-6-[(propan-2-yl)carbamoyl]-2,3-dihydrospiro[[1]benzopyran-4,1′-cyclopropane]-2′-carboxylate]+[ethyl (1′R,2′R)-6-[(propan-2-yl)carbamoyl]-2,3-dihydrospiro[[1]benzopyran-4,1′-cyclopropane]-2′-carboxylate])=97.5/2.5, whereby compound (3) was obtained in a high yield.

The method for producing the Compound (I) is preferably the method that the above described examples of 3. Second Alternative Preparation of Compound (4), 5. Alternative Preparation of Compound (4a), 6. Preparation of Compound (6), 8. Alternative Preparation of Compound (7), 12. Third Alternative Preparation of Compound (5), 15. Second Alternative Preparation of Compound (3), 18. Alternative Preparation of Compound (2) Using Compound (4a), and 21. Second Alternative Preparation of Compound (I) are combined.

It is to be appreciated that the Detailed Description section, and not the Summary and Abstract sections, is intended to be used to interpret the claims. The Summary and Abstract sections can set forth one or more but not all exemplary embodiments of the present disclosure as contemplated by the inventor(s), and thus, are not intended to limit the present disclosure and the appended claims in any way.

The present disclosure has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the disclosure that others can, by applying knowledge within the skill of the art, readily modify and/or adapt for various applications such specific embodiments, without undue experimentation, without departing from the general concept of the present disclosure. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary embodiments, but should be defined only in accordance with the following claims and their equivalents.

1. A process for preparing a compound of formula (2): 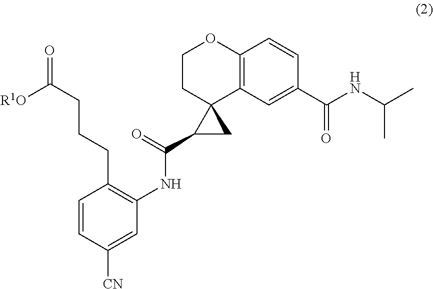
wherein R1 is a C1-C6 alkyl group or a benzyl group, the process comprising:
reacting a compound of formula (3): 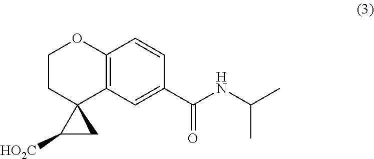 [SMILES: CC(C)NC(=O)c1ccc2c(c1)[C@]1(CCO2)C[C@H]1C(=O)O]
with a compound of formula (4a) or a salt thereof: 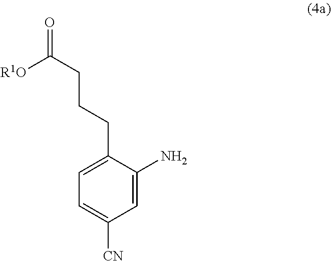
wherein R¹ has the same meaning as defined above.

2. The process of claim 1, wherein the reaction of the compound of formula (3) with the compound of formula (4a) or a salt thereof is conducted in the presence of a coupling agent.

3. The process of claim 1, wherein the compound of formula (3) is reacted with the compound of formula (4a).

4. The process of claim 2, wherein the coupling agent is selected from the group consisting of N,N,N′,N′-tetramethylchloroformamidinium hexafluorophosphate, 2,4,6-tripropyl-1,3,5,2,4,6-trioxatriphosphorinane-2,4,6-trioxide, diphenyl phosphoryl chloride, and a combination thereof.

5. (canceled)

6. The process of claim 1, further comprising:
treating a compound of formula (14): 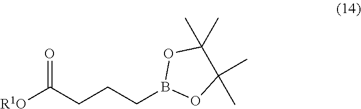
with a compound of formula (11): 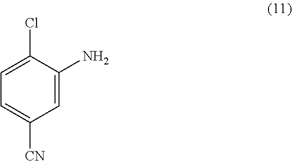
in the presence of a second palladium catalyst and a second ligand to provide the compound of formula (4a) or a salt thereof, wherein the second palladium catalyst and the second ligand are optionally in a form of a complex thereof.

7-10. (canceled)

11. The process of claim 6, wherein the second palladium catalyst is selected from the group consisting of tetrakis(triphenylphosphine)palladium(0) (Pd(PPh₃)₄), palladium acetate, allyl palladium chloride dimer, and a combination thereof.

12. (canceled)

13. The process of claim 6, wherein the second ligand is selected from the group consisting of 1,1-bis(dicyclohexylphosphino)ferrocene, 2-(dicyclohexylphosphino)3,6-dimethoxy-2′,4′,6′-triisopropyl-1,1′-biphenyl, di(1-adamantyl)-n-butylphosphine, 1,2-ethanediylbis[dicyclohexyl]phosphine, and a combination thereof.

14. (canceled)

15. The process of claim 6, wherein the second palladium catalyst and the second ligand are employed as a complex thereof.

16. The process of claim 15, wherein the complex is [1,1′-bis(dicyclohexylphosphino) ferrocene]dichloropalladium(II).

17-34. (canceled)

35. The process of claim 1, further comprising,
reacting a compound of formula (5): 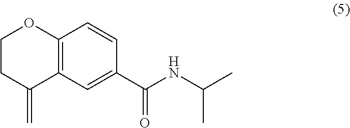 [SMILES: C=C1CCOc2ccc(C(=O)NC(C)C)cc21]
with a cyclopropanating agent in the presence of a catalyst and a chiral ligand to provide a compound of formula (5a): 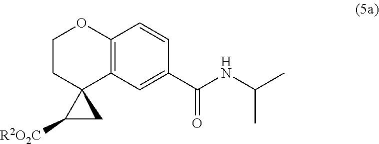
wherein R² is a C₁-C₆ alkyl group or a benzyl group; and
hydrolyzing the compound of formula (5a).

36. The process of claim 35, wherein the cyclopropanating agent is selected from the group consisting of ethyl diazoacetate, methyl diazoacetate, n-butyl diazoacetate, benzyl diazoacetate, isopropyl diazoacetate, t-butyl diazoacetate, and a combination thereof.

37. The process of claim 35, wherein the catalyst is selected from the group consisting of dichloro(p-cymene)ruthenium(II) dimer, dibromo(p-cymene)ruthenium(II) dimer,diiodo(p-cymene)ruthenium(II) dimer, and a combination thereof.

38. The process of claim 35, wherein the chiral ligand is selected from the group consisting of (S,S)-2,2′-(2,6-pyridinediyl)bis(4-isopropyl-2-oxazoline), (−)-2,6-bis[(3aS,8aR)-3a,8a-dihydro-8H-indeno[1,2-d]oxazolin-2-yl]pyridine,2,6-bis[(4R,5R)-4-methyl-5-phenyl-2-oxazolinyl]pyridine, and a combination thereof.

39. The process of claim 35, wherein the hydrolysis is conducted in the presence of a hydrolyzing agent selected from the group consisting of sodium hydroxide, lithium hydroxide, potassium hydroxide, tetraethylammonium hydroxide, potassium trimethylsilanolate, tetramethylammonium hydroxide, tetra-n-propylammonium hydroxide, tetra-n-butylammonium hydroxide, benzyltrimethylammonium hydroxide, and a combination thereof.

40. The process of claim 35, further comprising:
treating a compound of formula (7): 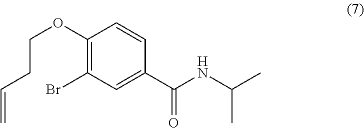 [SMILES: C=CCCOc1ccc(C(=O)NC(C)C)cc1Br]
with a catalyst in the presence of a ligand to provide the Compound (5).

41. The process of claim 40, wherein the catalyst is selected from the group consisting of palladium acetate, allyl palladium chloride dimer, and a combination thereof.

42. The process of claim 40, wherein the ligand is selected from 1,2-bis(diphenylphosphino)benzene, 2-dicyclohexylphosphino-2′,6′-diisopropoxy-1,1′-biphenyl, (4-(N,N-dimethylamino)phenyl)di-tert-butyl phosphine, and butyl di-1-adamantylphosphine.

43. The process of claim 40, further comprising:
treating a compound of formula (6): 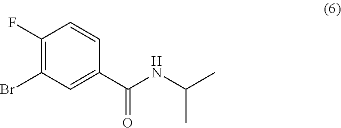
with 3-buten-1-ol to provide the compound of formula (7).

44. A process for preparing a compound of formula (I): 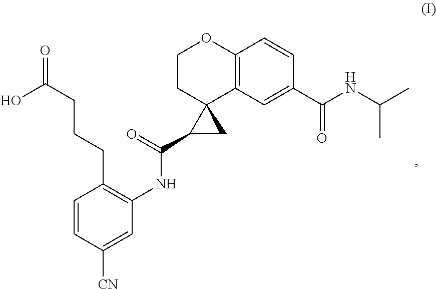 [SMILES: CC(C)NC(=O)c1ccc2c(c1)[C@]1(CCO2)C[C@H]1C(=O)Nc1cc(C#N)ccc1CCCC(=O)O]
comprising reacting a compound of formula (3): 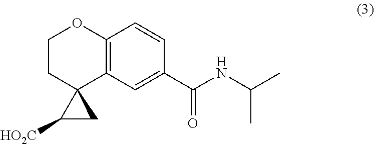 [SMILES: CC(C)NC(=O)c1ccc2c(c1)[C@]1(CCO2)C[C@H]1C(=O)O]
with a compound of formula (4a) or a salt thereof: 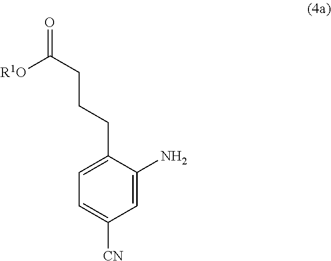
wherein R1 is a C1-C6 alkyl group or a benzyl group, to give a compound of formula (2): 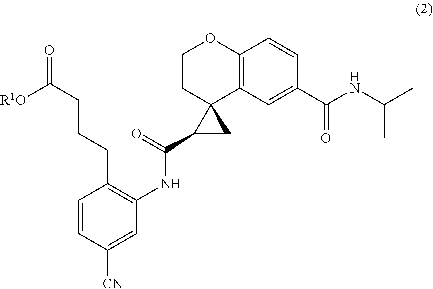
wherein R1 is a C1-C6 alkyl group or a benzyl group; and
hydrolyzing the compound of formula (2) to provide the compound of formula (I).

45. A compound of (1'S,2′R)-6-[(propan-2-yl)carbamoyl]-2,3-dihydrospiro[[1]benzopyran-4,1′-cyclopropane]-2′-carboxylic acid.
